(19) United States
(12) Patent Application Publication
(10) Pub. No.: US 2025/0281528 A1
(43) Pub. Date: Sep. 11, 2025

(54) GRAPHENE OXIDE-CATIONIC SILVER NANOCOMPOSITES AND THEIR USE AS BROAD-SPECTRUM ANTIMICROBIAL AGENTS

(71) Applicant: Zentek Ltd., Guelph (CA)

(72) Inventors: Seyyedarash Haddadi, Guelph (CA); Colin Van Der Kuur, Kelowna (CA); Joseph Korkis, Burlington (CA); Deepak Sridhar, Guelph (CA)

(21) Appl. No.: 18/259,010

(22) PCT Filed: Dec. 20, 2021
(86) PCT No.: PCT/CA2021/051849

§ 371 Date: Jun. 22, 2023

Related U.S. Application Data
(60) Provisional application No. 63/128,993, filed on Dec. 22, 2020.
(60) Provisional application No. 63/161,873, filed on Mar. 16, 2021.

(30) Foreign Application Priority Data
Sep. 20, 2021 (WO) PCT/CA2021/051308

(51) Int. Cl. A61K 33/38; A61K 9/51; A61P 31/04; A61P 31/10; A61P 31/14

(52) U.S. Cl. CPC A61K 33/38; A61K 9/5115; A61P 31/04; A61P 31/10; A61P 31/14

(57) ABSTRACT

The present invention relates to antimicrobial compositions comprising graphene-silver cation nanocomposites and uses for same as a broad-spectrum antimicrobial agent, and uses for same for treating microbial infections, including infections by antimicrobial resistant and/or multidrug resistant pathogens.

### FIELD OF THE INVENTION

The present invention relates to the field of antimicrobial agents and, in particular, to graphene-silver nanocomposites and therapeutic and prophylactic compositions thereof to treat and/or prevent a broad-spectrum of microbial infections including against antimicrobial resistant (AMR) pathogens.

### BACKGROUND OF THE INVENTION

Microorganisms (or microbes) are single cell, cell cluster, or multicellular microscopic (or macroscopic) organisms including but not limited to, bacteria, fungi, and viruses. Pathogenic microbes have the potential to cause a multitude of infectious diseases through various modes of transmission including by contact, touch, or airborne transmission. For example, contamination of surfaces with one or more types of microorganisms, the transfer of microorganisms between surfaces, and/or the aerosol transfer of microbes in the air, can lead to transmission of illness and disease.

Infectious diseases caused by pathogenic microbes continue to be a global issue. For example, in recent years, there have been widespread outbreaks of Swine Flu, Ebola virus, Zika virus, norovirus, severe acute respiratory syndrome (SARS), Middle East respiratory syndrome (MERS), and most recently coronavirus disease 19 (COVID-19) which has been declared a pandemic by the World Health Organization.

Antimicrobial agents are appropriate treatment for acute, severe, persistent, or progressive infectious diseases and conditions. The efficacy of treatment depends on the accuracy of the diagnosis of infection and the appropriateness of the antimicrobial agent for the causative microorganism. However, even when infection is clinically apparent, the causative microorganism cannot always be identified, and empiric treatment with broad-spectrum agents is often necessary in many cases of serious disease.

Antimicrobial Resistance (AMR) occurs when bacteria, viruses, fungi and parasites change over time and no longer respond to antimicrobial agents. As a result, antibiotics and other antimicrobial medicines become ineffective and infections become increasingly difficult or impossible to treat, increasing the risk of disease spread, severe illness, and death. The rapid emergence of resistant pathogens is occurring worldwide, endangering the efficacy of existing antimicrobial agents.

The AMR pathogens that pose the greatest threat to human health are generally referred to as ESKAPE pathogens. The ESKAPE pathogens encompass six pathogens with growing antimicrobial resistance and virulence: Enterococcus faecium, Staphylococcus aureus, Klebsiella pneumoniae, Acinetobacter baumannii, Pseudomonas aeruginosa, and Enterobacter spp. The World Health Organization (WHO) has also listed ESKAPE pathogens in the list of “priority pathogens” against which new antimicrobial agents are urgently needed. These pathogens have been categorized as critical, high and medium priority, according to the urgency of need for new antimicrobials. In particular, the critical category includes: Acinetobacter baumannii, carbapenem-resistant; Pseudomonas aeruginosa, carbapenem-resistant; and Enterobacteriaceae, carbapenem-resistant, ESBL-producing. The high priority category includes: Enterococcus faecium, vancomycin-resistant; Staphylococcus aureus, methicillin-resistant, vancomycin-intermediate and resistant; Helicobacter pylori, clarithromycin-resistant; Campylobacter spp., fluoroquinolone-resistant; Salmonellae, fluoroquinolone-resistant; and Neisseria gonorrhoeae, cephalosporin-resistant, fluoroquinolone-resistant. The medium priority category has been identified by the WHO as including: Streptococcus pneumoniae, penicillin-non-susceptible; Haemophilus influenzae, ampicillin-resistant; and Shigella spp., fluoroquinolone-resistant.

The most critical group identified by the WHO includes multidrug resistant (MDR) organisms that pose a particular threat in hospitals, nursing homes, and among patients whose care requires devices such as ventilators and blood catheters. These organisms include, for example, Acinetobacter, Pseudomonas and various Enterobacteriaceae (including Klebsiella, E. coli, Serratia, and Proteus). These pathogens can cause severe and often deadly infections such as bloodstream infections and pneumonia. Moreover, these pathogens have become resistant to a large number of antimicrobial agents, including the best available antibiotics for treating MDR pathogens, for example, carbapenems and third generation cephalosporins. The second and third categories of AMR pathogens identified by the WHO as being high and medium priority pathogens, include other increasingly drug-resistant organisms that cause more common diseases such as gonorrhoea and food poisoning caused by salmonella.

The emergence and spread of AMR pathogens continues to threaten the ability to treat microbial infections, however, the clinical pipeline of new antimicrobials is failing to keep up. Given the diminishing effectiveness of existing antimicrobial agents and the exceedingly urgent need to develop new antimicrobial compositions, a continuing need exists for novel therapeutic and prophylactic modalities to treat and/or prevent common and recurrent microbial infections, and to address the risk of AMR pathogens.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide graphene-silver nanocomposites and uses for same as a broad-spectrum antimicrobial. In accordance with one aspect of the invention, there is described an antimicrobial nanocomposite comprising graphene oxide (GO) with cationic silver (Ag⁺) moieties respectively bound to the GO.

In accordance with another aspect of the invention, there is described an antimicrobial nanocomposite comprising GO and Ag⁺ bound in a complex to the GO.

In accordance with another aspect of the invention, there is described an antimicrobial nanocomposite comprising GO and Ag⁺ bound in a complex to the GO, effective at killing microbial pathogens.

In accordance with another aspect of the invention, there is described an antimicrobial nanocomposite comprising GO and Ag⁺ bound in a complex to the GO, effective at killing antimicrobial resistant (AMR) microbial pathogens.

In accordance with another aspect of the invention, there is described an antimicrobial nanocomposite comprising GO and Ag⁺ bound in a complex to the GO, effective at killing multidrug resistant (MDR) microbial pathogens.

In accordance with another aspect of the invention, there is described an antimicrobial composition comprising a GO-Ag⁺ nanocomposite and one or more pharmaceutically acceptable carriers, diluents and/or excipients.

In accordance with another aspect of the invention, there is described a composition comprising a therapeutically effective amount of an antimicrobial nanocomposite comprising GO and Ag⁺ bound in a complex to the GO, for preventing or treating a microbial infection or disease.

In accordance with another aspect of the invention, there is described a method for preventing or treating a microbial infection selected from bacterial infection, fungal infection, viral infection, or any combination thereof, comprising administering to a subject in need thereof an antimicrobial nanocomposite comprising GO and Ag⁺ bound in a complex to the GO.

In accordance with another aspect of the invention, there is described the use of an antimicrobial nanocomposite comprising GO and Ag⁺ bound in a complex to the GO, in the manufacture of a medicament for preventing or treating a microbial infection selected from bacterial infection, fungal infection, viral infection, or any combination thereof.

In accordance with another aspect of the invention, there is described a method for preventing or treating a respiratory tract infection in a subject comprising administering to the subject an effective amount of a composition comprising a therapeutically effective amount of an antimicrobial nanocomposite comprising GO and Ag⁺ bound in a complex to the GO.

In accordance with another aspect of the invention, there is described a composition comprising a therapeutically effective amount of an antimicrobial nanocomposite comprising GO and Ag⁺ bound in a complex to the GO, for treating a multidrug resistant microbial infection in a subject.

In accordance with another aspect of the invention, there is provided a method for treating a multidrug resistant microbial infection in a subject comprising administering to the subject an effective amount of a composition comprising a therapeutically effective amount of an antimicrobial nanocomposite comprising GO and Ag⁺ bound in a complex to the GO.

In accordance with another aspect of the invention, there is provided a kit comprising a container having contained therein a composition comprising a therapeutically effective amount of an antimicrobial nanocomposite comprising GO and Ag⁺ bound in a complex to the GO, the container adapted to deliver the composition by an intranasal or pulmonary route.

### BRIEF DESCRIPTION OF THE DRAWINGS

These and other features of the invention will become more apparent in the following detailed description in which reference is made to the appended drawings.

FIG. 1 is a schematic illustration of Ag⁺ complex formation with oxygen-containing functional groups of GO and the formation of Ag nanoparticles on the surface of GO nanosheets.

FIGS. 2A to 2D are results of XPS and AES analysis of a sample according to an embodiment of the present invention.

FIG. 3 is a chart showing a comparison of chemical states between silver and silver nano-composites formed.

FIGS. 4A to 4F are SEM (SE) and SEM (Backscattered electron image/Z count) images of GO before contact with silver cations.

FIGS. 5A to 5C are EDS images and results charts of GO before contact with silver cations.

FIGS. 6A to 6F are SEM (SE) and SEM (Z count) images of GO after contact with silver cations but before purification.

FIGS. 7A and 7B are EDS images and elemental results of GO after contact with silver cations but before purification.

FIGS. 8A to 8D are SEM (SE) and SEM (Z count) images of GO after contact with silver cations and after purification.

FIGS. 9A and 9B are EDS images and elemental results of GO after contact with silver cations and after purification.

FIG. 10 presents plasma concentration time profiles of Ag for Day 1 of daily oral dosing of GO-Ag⁺ nanocomposite in female and male rats (▪50 mg/kg/day, ▴250 mg/kg/day, ●1000 mg/kg/day).

FIG. 11 presents plasma concentration time profiles of Ag for Day 7 of daily oral dosing of GO-Ag⁺ nanocomposite in female and male rats (▪50 mg/kg/day, ▴250 mg/kg/day, ●1000 mg/kg/day).

### DETAILED DESCRIPTION OF THE INVENTION

Two-dimensional graphene oxide (GO) has shown promise as a nanomaterial for various applications, including biomedical applications, due to its lateral size and the colloidal properties of the nanosheets. The antibacterial effect of GO has also been described, specifically as a supporting and stabilizing agent for antibacterial compounds, for example, silver nanoparticles (Ag NPs). More specifically, GO-AgNP nanocomposites have been thought to enhance the antibacterial effect of AgNPs by immobilizing the AgNPs on GO to prevent the movement of the nanoparticles.

While GO-AgNP nanocomposites have been shown to have antibacterial effects, the use of GO-AgNP nanocomposites for broad-spectrum antimicrobial applications has been shown to have a number of limitations, including for example its instability in water affecting the ability of GO-AgNP to effectively release the antimicrobially active form of Ag⁺ ions. As a result, additional chemicals, solvents, and post-processing, of the GO and/or the Ag components, are typically required to stabilize GO-AgNP nanocomposites for use, for example, capping agents, thiol functionalization, or co-reduction of the GO or AgNP are typically required for preparing the GO-AgNP nanocomposites.

According to certain embodiments of the present invention, GO-Ag⁺ nanocomposites that comprise GO nanosheets with cationic silver (Ag) moieties bound to the GO are described that are stable in water without the need for additional chemicals, solvents or post-processing.

According to certain embodiments of the present invention, GO-Ag⁺ nanocomposites are described that unexpectedly exhibit potent antibacterial as well as antifungal and/or antiviral efficacy. Moreover, in further embodiments, GO-Ag⁺ nanocomposites are described that unexpectedly exhibit potent antimicrobial efficacy against AMR and/or MDR pathogens. Without being bound by theory, it is contemplated that GO produces a high surface area for the attachment of Ag⁺ cations and further stabilizes the Ag⁺ cations on the GO-Ag⁺ nanocomposite. In this way, the GO nanosheets provide a stabilizing framework for the Ag⁺ cations which cause oxidative stress to the pathogen. Additionally, the functional oxygen groups on GO in combination with the physical shearing effect of the sharp edges of the graphene on a pathogen, are further believed to work synergistically with the antimicrobial effect of the Ag⁺ cations. Specifically, it is believed that GO envelopes and captures the pathogen due to its affinity to carbon, and further pierces the cell membrane to deliver oxidative stress to the pathogen due to the Ag⁺.

According to embodiments, the in-situ bonding of cationic silver (Ag) to GO facilitates a uniformly distributed complex, unlike the mixing of pre-formed nanoparticles with GO which often forms agglomerations. This provides enhanced surface area of the formed active material and an ability to effectively use Ag⁺ to create oxidative stress to the pathogen. According to embodiments, the GO and Ag⁺ components of the GO-Ag⁺ nanocomposites have a synergistic antimicrobial efficacy. In certain embodiments, the GO-Ag⁺ nanocomposites exhibit broad spectrum antimicrobial efficacy at a concentration less than 0.1 μg/mL, 0.08 μg/mL, 0.06 μg/ml, 0.04 μg/mL, 0.02 μg/mL, 0.009 μg/mL, or 0.007 μg/mL.

In certain embodiments, the GO-Ag⁺ nanocomposites comprise a nanocomposite formed by graphene oxide (GO) sheets with silver cation (Ag⁺) moieties bound to the GO. In further embodiments, the silver cation (Ag⁺) moieties are bound to the GO as complexes. In further embodiments, the nanocomposite comprises GO sheets with a combination of Ag⁺ moieties bound to the GO by complex bonds, as well as silver nanoparticles (Ag NP) chemically bonded and in some cases physically adsorbed onto the GO thereto. According to certain embodiments, the majority of the attached silver is in the cationic form (Ag⁺) and is bound to the GO. According to some embodiments, the majority of the attached silver is in the cationic form and bound to the GO as complexes. In further embodiments, the majority of the attached silver is in the cationic form and bound to the GO as complexes and further comprises a small amount of silver nanoparticles. The silver nanoparticles are formed due to reduction of the cationic form that accompanies the oxidation of certain functional groups on the GO. Such functional groups may include epoxy, hydroxyl, carboxylic, carbonyl, quinone or any specific functional group introduced on the surface during the preparation of GO. In certain embodiments, the Ag⁺ may be attached to the GO surface by the addition of functional groups on the GO, including for example N or P functional groups. According to certain embodiments described herein, the GO-Ag⁺ nanocomposites comprise complexed GO-Ag⁺. In other embodiments, the GO-Ag⁺ nanocomposites comprise complexed GO-Ag⁺ and free Ag⁺ cations. According to further embodiments described herein, the GO-Ag⁺ nanocomposites comprise Ag in various chemical states such as Ag(0) or Ag(1) in salt, oxide or metal form. In certain embodiments, 50-98% of the attached silver is bonded to the GO by complex bonds. According to other embodiments, 65-85% of the attached silver is bonded by complex bonds. In further embodiments, 70% of the attached silver is bonded to the GO by complex bonds. In other embodiments, 85%-95% of the attached silver is bonded to the GO by complex bonds. In further embodiments, 90-95% of the attached silver is bonded to the GO by complex bonds.

In further embodiments, the GO sheets of the present invention have been made to have a sufficiently large surface area for other antimicrobial agents to be additionally combined, for example, the addition of one or more other metals such as Cu²⁺ and/or Zn²⁺ and/or Au²⁺ cations, or a combination thereof, to the GO-Ag⁺ nanocomposite. According to certain embodiments, the GO-Ag⁺ nanocomposite further includes one or more other metal and metal-ligand complexes, for example, copper (e.g., copper ion, copper nanoparticles), gold (gold ion, gold nanoparticles), and/or zinc (zinc ion, zinc nanoparticles), or any combination thereof.

According to embodiments of the invention, the GO-Ag⁺ nanocomposite compositions provide broad spectrum antimicrobial activity. In some embodiments, the described GO-Ag⁺ nanocomposite compositions have potent antimicrobial activity against a bacterial pathogen, a viral pathogen, or a fungal pathogen, or any combination thereof. In further embodiments, the GO-Ag⁺ nanocomposite compositions have antimicrobial activity against antimicrobial-resistant and/or multidrug-resistant microbial strains.

According to embodiments described herein, GO-Ag⁺ nanocomposites that comprise Ag⁺ cations unexpectedly exhibit a synergistic antimicrobial effect to accelerate the time to microbial death at low concentrations.

In addition, according to further embodiments, the described GO-Ag⁺ nanocomposite compositions unexpectedly do not induce significant toxicity when administered at the effective dose levels to subjects. In some embodiments, the GO-Ag⁺ nanocomposites exhibited low to negligible toxicity at dosage ranges of up to 50, 250, and 1000 mg/kg/day. Accordingly, in certain embodiments, the present invention provides for the use of GO-Ag⁺ nanocomposite compositions to treat an established infection in a subject, for example, an infection with a bacterial pathogen, a viral pathogen, or a fungal pathogen. In further embodiments, the present invention provides for the use of GO-Ag⁺ nanocomposite compositions to treat one or more of gram positive bacteria, gram negative bacteria, aerobic bacteria, anaerobic bacteria, and/or yeast. In additional embodiments, the present invention provides for the use of GO-Ag⁺ nanocomposite compositions to treat antimicrobial resistant and/or multidrug resistant microbial pathogens.

According to particular embodiments, the present invention provides for the use of GO-Ag⁺ nanocomposite compositions to treat a respiratory infection, for example, an upper respiratory infection (URI) or a lower respiratory infection (LRI).

Drug delivery by aerosol inhalation is a well-established procedure in the treatment of respiratory infections. The use of inhaled aerosols provides for a route of direct delivery of a therapeutic to the respiratory tract thereby allowing for selective treatment of the target area in respiratory infections, while minimizing the need for systemic administration and consequent risk of drug resistance and side effects. Further advantages for drug delivery by aerosol inhalation include the use of smaller doses than those given orally, relatively rapid onset of the therapeutic effect, and minimized systemic adverse effects.

In addition to efficacy of the active ingredient being inhaled, success of aerosol inhalation delivery of a therapeutic is dependent on the site and extent of deposition of the active ingredient in the respiratory tract. In particular, spatial distribution of deposited particles and, as a consequence, drug efficiency is strongly affected by particle size. Large particles (>6 μm) tend to mainly deposit in the upper airway, limiting the amount of drugs that can be delivered to the lung. Small particles (<2 μm) deposit mainly in the alveolar region and are probably the most apt to act systemically, whereas the particle in the size range 2-6 μm are best suited to treat the central and small airways (C. Darquenne, Aerosol Deposition in Health and Disease. J Aerosol Medicine and Pulmonary Drug Delivery 25:3:140-147 (2012)).

According to embodiments of the present invention, the GO-Ag⁺ nanocomposite particles can be made to range in size from <2 μm up to 10 μm. In certain embodiments, the GO-Ag⁺ nanocomposite particles can be prepared for aerosol inhalation delivery to the respiratory tract and range in size from 2-10 μm. In further embodiments, the GO-Ag⁺ nanocomposite particles can be prepared to range in size from 2-6 μm. In other embodiments, the GO-Ag⁺ nanocomposite particles can be prepared for aerosol inhalation delivery to the respiratory tract to act systemically. According to such embodiments, the GO-Ag⁺ nanocomposite particles can be prepared to be <2 μm. In other embodiments, the GO-Ag⁺ nanocomposite particles can be prepared to range in size from 400 nm to 1 μm. In further embodiments, the GO-Ag⁺ nanocomposite particles can be prepared to range in size from 750 nm to 1 μm.

### Definitions

Unless defined otherwise, all technical and scientific terms used herein have the same meaning as commonly understood by one of ordinary skill in the art to which this invention belongs.

As used herein, the term “about” refers to an approximately +/−10% variation from a given value. It is to be understood that such a variation is always included in any given value provided herein, whether or not it is specifically referred to.

The use of the word “a” or “an” when used herein in conjunction with the term “comprising” may mean “one,” but it is also consistent with the meaning of “one or more,” “at least one” and “one or more than one.”

As used herein, the word “antimicrobial” means the destruction and/or inactivation of a pathogenic microorganism/microbe.

The use of the word “antiviral” and “virucidal” may be used interchangeably herein to mean the destruction and/or inactivation of a virus.

The use of the word “antibacterial” and “bactericidal” may be used interchangeably herein to mean the destruction and/or inactivation of bacteria.

The use of the word “antifungal” and “fungicidal” may be used interchangeably herein to mean the destruction and/or inactivation of a fungus.

As used herein, the words “comprising” (and grammatical variations thereof, such as “comprise” and “comprises”), “having” (and grammatical variations thereof, such as “have” and “has”), “including” (and grammatical variations thereof, such as “includes” and “include”) or “containing” (and grammatical variations thereof, such as “contains” and “contain”) are inclusive or open-ended and do not exclude additional, unrecited elements or method steps.

The terms “attenuate”, “inhibit”, “prevent”, “treat”, and grammatical variations thereof, as used herein, refer to a measurable decrease in a given parameter or event.

The term “treatment method”, or “method for the treatment of a pathology or disorder”, means therapy aimed at restoring the health condition of a subject, maintaining the existing condition and/or preventing the worsening of said health condition.

The term “pathogen,” as used herein, refers to an organism capable of causing an infection, disease, or disorder in a host including, but not limited to, bacteria, viruses, protozoa, fungi and parasites.

The term “subject” or “patient” as used herein refers to an animal in need of treatment.

The term “animal,” as used herein, refers to both human and non-human animals, including, but not limited to, mammals, birds and fish, and encompasses domestic, farm, zoo, laboratory and wild animals, such as, for example, cows, pigs, horses, goats, sheep or other hoofed animals, dogs, cats, chickens, ducks, non-human primates, guinea pigs, rabbits, ferrets, rats, hamsters and mice.

Administration of graphene-silver nanocomposite compositions “in combination with” one or more further therapeutic agents is intended to include simultaneous (concurrent) administration and consecutive administration. Consecutive administration is intended to encompass various orders of administration of the therapeutic agent(s) and the composition of the invention to the subject with administration of the therapeutic agent(s) and the composition being separated by a defined time period that may be short (for example in the order of minutes) or extended (for example in the order of days or weeks).

The term “Minimum Inhibitory Concentration” or “MIC”, as used herein, refers to the lowest concentration of an antimicrobial compound/agent that reduces the viability of the initial microbial inoculum by ≥99.9%.

The term “antimicrobial resistance” or “antimicrobial resistant”, as used herein, refers to a pathogen that is resistant to one or more antimicrobial agent.

The term “multidrug resistant” or “MDR”, as used herein, refers to a pathogen that is resistant to more than one antimicrobial agent, drug, or medicament.

The term “complex bond” as used herein, also known as an acid-base Lewis interaction, a coordinate bond or chelated bond or co-ordinate covalent bond, forms a coordination compound in which a silver ion is attached by coordinate covalent bonds to the GO. The bonding in a complex or chelate bond occurs because the oxygen groups of the GO have at least 2 pairs of unshared electrons; and both the electrons involved in the bonding comes from this ligand These pairs of unshared electrons are regions of negative electrical charge to which are attracted the silver cations. If only 2 pairs of unshared electrons form a complex with silver, this is known as a bidentate arrangement. If 3 pairs of unshared electrons form a complex with silver, this is known as a tridentate arrangement.

It is contemplated that any embodiment discussed herein can be implemented with respect to any method of composition of the invention, and vice versa. Furthermore, compositions of the invention can be used to achieve the methods of the invention.

### Preparation of Graphene Oxide/Silver Cation Nanocomposites

### Graphene Oxide

Graphene Oxide (GO) is a two dimensional form of carbonaceous material that has oxygen-containing groups added to its edges and basal planes including epoxide, carboxyl and hydroxyl groups. GO is known to have a large surface area and exhibits no corrosive characteristics. GO can be synthesized by standard techniques known in the art, for example, Staudenmaier, Hofmann, Brodie, Hummers, and electrochemical exfoliation, which are methods that involve mechanical or thermal exfoliation, chemical vapour deposition (CVD), and epitaxial growth.

According to certain embodiments, the GO can be synthesized by any of the standard methods known in the art. In certain embodiments, the GO can be synthesized by electrochemical exfoliation. In further embodiments, the GO can be synthesized by the Hummers' method.

According to further embodiments, the GO can be synthesized by a modified version of the Hummers' method. In certain embodiments, the GO is synthesized by a modified version of the Hummers' method in which phosphoric acid is eliminated from the process steps. It has unexpectedly been found that by eliminating the use of phosphoric acid from the synthetic process, fewer chemicals are required and synthesis is more efficient, and involving fewer steps.

In preferred embodiments, the GO is synthesized to maximize the available surface area for attachment of ionic silver. According to such embodiments, the method is adapted for the synthesis of smaller graphene flakes to produce GO having increased surface area. In certain embodiments, for example, the GO flake size is reduced through sonication. In certain embodiments, the graphene flakes have a particle size ranging from 50 nm to 5 μm. In further embodiments, the graphene flakes have a particle size ranging from 50 nm to 100 nm, 100 nm to 200 nm, 200 nm to 400 nm, 400 nm to 500 nm, 500 nm to 5 μm, 750 nm to 4 μm. In additional embodiments, the graphene flakes have a particle size ranging from 1 to 3 μm.

In various embodiments, the GO has an oxygen content between 5 to 40%. In further embodiments, the GO has an oxygen content of between 10 to 35%. In additional embodiments, the GO has an oxygen content between 15 to 25%. In further embodiments, the GO has an oxygen content of between 20 to 25%. In other embodiments, the GO has an oxygen content between 20 to 35%. In additional embodiments, the GO has an oxygen content between 28 to 35%.

In various embodiments, the GO has between 1 to 10 layers with a d-spacing that ranges between 0.3 nm to 1 nm. In certain embodiments, the GO has between 1 to 10 layers with a d-spacing that ranges between 0.3 to 0.8 nm. In further embodiments, the GO has between 1 to 10 layers with d-spacing that is at least about 0.8 nm. In additional embodiments, the GO has between 1 to 10 layers with d-spacing that is at least about 0.4 nm.

In certain embodiments, the GO has at least 3 layers. In further embodiments the GO has at least 4 layers. In additional embodiments, the GO has at least 5 layers. In other embodiments, the GO has at least 7 layers.

### Silver Cations

Silver nanoparticles (AgNPs) have been widely studied as an antimicrobial agent, however, the effectiveness of AgNPs for inactivating various types of bacteria and viruses is limited by the ability of AgNPs to release silver ions. The release rate of Ag⁺ from AgNPs to interact directly with phosphorus- or sulfur-containing biomolecules, including DNA, RNA, and proteins, affects the antimicrobial efficacy of AgNPs. In particular, the size, shape, and concentration of AgNPs have been identified as limiting factors that affect their antimicrobial capabilities.

Accordingly, various embodiments described herein relate to the attachment of silver cations (Ag⁺) to GO. In certain embodiments, the GO comprises between 3-25% w/w of silver in its cationic form. In other embodiments, the concentration of cationic silver bonded to GO is between 5-15% w/w. In further embodiments, the concentration of cationic silver bonded to GO is between 10-20% w/w. According to further embodiments, the concentration of cationic silver bonded to GO is between 3-10% w/w. In other embodiments, the concentration of cationic silver bonded to GO is between 4-8% w/w.

According to further embodiments the GO-Ag⁺ nanocomposite also includes colloidal silver, i.e., AgNPs. In such embodiments, the GO-Ag⁺/AgNP nanocomposite can comprise these two forms of silver in ratios of Ag⁺:AgNP of 500:1, 400:1, 300:1, 200:1, 100:1, 90:1, 80:1, 70:1, 60:1, 50:1, 40:1, 30:1, 20:1, 10:1, and 5:1. In certain embodiments, the ratio of Ag⁺:AgNP bonded to the GO nanocomposite is 50:1. According to further embodiments, the ratio of Ag⁺:AgNP bonded to the GO nanocomposite is 40:1. According to other embodiments, the ratio of Ag⁺:AgNP bonded to the GO nanocomposite is 20:1. According to certain embodiments, the ratio of Ag⁺:AgNP bonded to the GO nanocomposite is 15:1. In further embodiments, the ratio of Ag⁺:AgNP bonded to the GO nanocomposite is 10:1. According to certain embodiments, the ratio of Ag⁺:AgNP bonded to the GO nanocomposite is 11:1. In further embodiments, the ratio of Ag⁺:AgNP bonded to the GO nanocomposite is between 10:1 and 15:1.

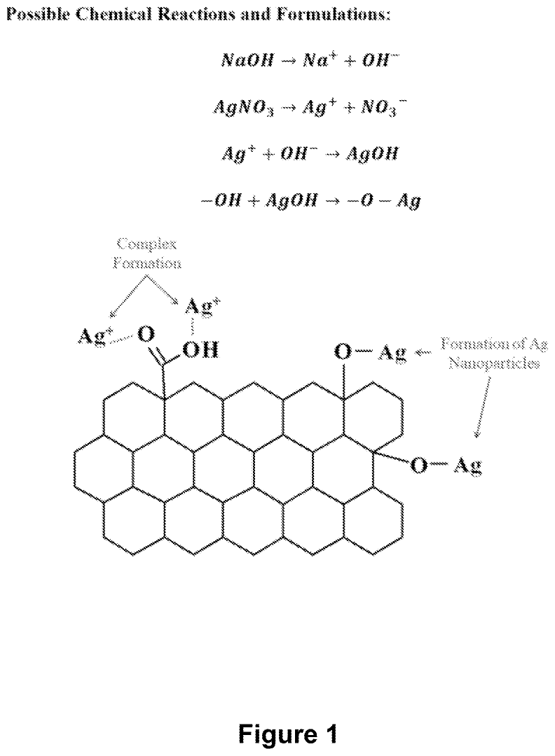

FIG. 1 illustrates the non-limiting reaction of the adhesion of ionic Ag⁺ and AgNPs to the GO contemplated herein.

### Other Metals

It is further contemplated that the GO-Ag⁺ nanocomposite described herein may also include additional metals. Illustrative but non-limiting nanocomposites described herein comprise GO-Ag⁺ nanocomposite and one or more other metals. Illustrative other metals include, but are not limited to copper (e.g., copper ion, copper nanoparticles), gold (gold ion, gold nanoparticles), and zinc (zinc ion, zinc nanoparticles).

In certain embodiments, the GO-Ag⁺ nanocomposite described herein are mixed with additives such as cations and nanomaterials or a combination of these. Cations include but not limited to Ag⁺, Cu²⁺, Zn²⁺. And metallic nanomaterials including but not limited to Ag, Cu, Zn. In further embodiments, these additives are chemically bonded to GO and in some cases physically adsorbed onto the GO thereto.

### Particle Size

The particle size of the GO-Ag⁺ nanocomposite may impact antimicrobial efficacy, particularly for aerosol inhalation delivery to the respiratory tract. According to embodiments of the present invention, the particle size of the GO-Ag⁺ nanocomposite may be made to range in size from <2 μm up to 10 μm. In certain embodiments, the GO-Ag⁺ nanocomposite particles can be prepared for aerosol inhalation delivery to the respiratory tract and range in size from 2-10 μm. In further embodiments, the GO-Ag⁺ nanocomposite particles can be prepared to range in size from 2-6 μm. In other embodiments, the GO-Ag⁺ nanocomposite particles can be prepared for aerosol inhalation delivery to the respiratory tract to act systemically. According to such embodiments, the GO-Ag⁺ nanocomposite particles can be prepared to be <2 μm. In other embodiments, the GO-Ag⁺ nanocomposite particles can be prepared to range in size from 50 nm to 5 μm. In further embodiments, the GO-Ag⁺ nanocomposite has a particle size ranging from 50 nm to 100 nm, 100 nm to 200 nm, 200 nm to 400 nm, 400 nm to 500 nm, 500 nm to 5 μm, 750 nm to 4 μm. In additional embodiments, the GO-Ag⁺ nanocomposite has a particle size ranging from 1 to 3 μm. In further embodiments, the GO-Ag⁺ nanocomposite particles can be prepared to range in size from 50 nm to 1 μm.

According to certain embodiments, the particle size of the GO-Ag⁺ nanocomposite can be established by controlling the size of the graphene flakes used as starting material for producing the GO. In further embodiments, the GO flake size can be further reduced through methods known in the art, for example, sonication, mechanical shearing, and/or water jet milling, followed by centrifugation, to reduce the size of the GO flakes to the desired particle size range for the particular application. The initial particle size can then be selected via an air classification system. Centrifugation can be used to remove larger flakes into the pellet while retaining smaller flake sizes in the supernatant. Repeated centrifugation can classify or separate the desired size flakes suitable for the particular application.

### Exemplary Method for Producing GO-Ag⁺ Nanocomposite

In accordance with one non-limiting aspect of the present invention, an antimicrobial nanocomposite is produced by a method comprising:
(a) synthesizing graphene oxide (GO), comprising the steps of:
   (i) mixing graphite powder with a 98% sulfuric acid solution at a volume-to-mass ratio of about 30 mL: 1 g of the sulfuric acid solution to the graphite powder to from a suspension;
   (ii) sonicating the suspension for 30 minutes at three, 6-hour intervals, at 50° C.;
   (iii) transferring the suspension to an ice-water bath and gradually adding potassium permanganate to the suspension, wherein the mass ratio of said potassium permanganate to graphite powder is about 4:1;
   (iv) stirring the mixture for up to 12 hours at 35° C. with intermittent sonication for 15 to 30 minutes after 8 hours;
   (v) reducing the temperature of the mixture to below 5° C. in an ice-bath and adding distilled water having a temperature of between 2-5° C. to the mixture, wherein the volume to mass ratio of the water to graphite being about 100 mL: 1 g;
   (vi) adding 30% hydrogen peroxide dropwise to the mixture until the mixture changes color from dark brown to yellow;
   (vii) sonicating the mixture for 30 minutes to accelerate the separation of GO nanosheets; and
   (viii) purifying the GO nanosheets by washing 3 times with 1 M hydrochloric solution followed by washing 3 times with a mixture of water and ethanol (8:2 v/v), wherein the mixture is sonicated for 30 minutes between each washing and the pH of the mixture adjusted to between 3 and 4 by the addition of 1 M potassium hydroxide solution; and
(b) fixing silver cations to the GO nanosheets, comprising the steps of:
   (i) sonicating the GO nanosheets in deionized water for 30 minutes, wherein the volume-to-mass ratio of the GO nanosheets to water is about 0.1 g: 30 mL, to form a suspension, and adjusting the pH of the suspension to 10 using a 0.1 M sodium hydroxide solution;
   (ii) adding a 10 M silver nitrate solution to the suspension, wherein the volume to mass ratio of the silver nitrate solution to GO nanosheets being about 0.1 g: 2 mL;
   (iii) adding deionized water to the suspension and stirring for 20 hours at 60° C. to reduce the viscosity of the solution, wherein the volume to mass ratio of the deionized water to GO nanosheets being about 0.1 g: 20 mL; and
   (iv) centrifuge-washing the suspension in deionized water 3 times to collect the graphene oxide-silver cation nanocomposite.

Without being limited to any particular theory, it is believed that GO silver cation nanocomposites are formed in part by a first silver cation monolayer being deposited and strongly bonded to the GO sheet, with successive layers of silver cations being weakly bonded primarily through physisorption.

### Antimicrobial Activity

In various embodiments, the GO-Ag⁺ nanocomposites described herein, and/or compositions or formulations comprising these nanocomposites, exhibit antimicrobial activity against a spectrum of microbial targets. In particular, it was unexpectedly found that the GO-Ag⁺ nanocomposites described herein exhibit a Minimum Inhibitory Concentration (MIC) of <1 μg/mL against a spectrum of microbial targets. The unexpectedly low MIC values exhibited across the spectrum suggest that the GO-Ag⁺ nanocomposites are effective antimicrobial agents with minimal risk of toxicity for animals and humans.

According to certain embodiments, the GO-Ag⁺ nanocomposites described herein exhibit an MIC of <1 μg/mL against microbial pathogens. In further embodiments, the GO-Ag⁺ nanocomposites described herein exhibit an MIC of <0.5 μg/mL against microbial pathogens. In other embodiments, the GO-Ag⁺ nanocomposites described herein exhibit an MIC of <0.25 μg/mL against microbial pathogens. In further embodiments, the GO-Ag⁺ nanocomposites described herein exhibit an MIC of <0.125 μg/mL against microbial pathogens. In other embodiments, the GO-Ag⁺ nanocomposites described herein exhibit an MIC of <0.0625 μg/mL against microbial pathogens. In further embodiments, the GO-Ag⁺ nanocomposites described herein exhibit an MIC of <0.031 μg/mL against microbial pathogens. In other embodiments, the GO-Ag⁺ nanocomposites described herein exhibit an MIC of <0.0156 μg/mL against microbial pathogens. In further embodiments, the GO-Ag⁺ nanocomposites described herein exhibit an MIC of <0.008 g/mL against microbial pathogens.

In certain embodiments, the GO-Ag⁺ nanocomposites described herein exhibit an antimicrobial efficacy against viral, bacterial, and/or fungal pathogens. In further embodiments, the GO-Ag⁺ nanocomposites described herein, and/or compositions or formulations comprising these nanocomposites, exhibit virucidal and/or antiviral effect against viral targets, including but not limited to, enveloped viruses such as herpesviruses, poxviruses, hepadnaviruses, asfarviridae, flavivirus, alphavirus, togavirus, coronavirus, hepatitis D, orthomyxovirus, paramyxovirus, rhabdovirus, bunyavirus, filovirus, retroviruses. According to further embodiments, the GO-Ag⁺ nanocomposite compositions exhibit virucidal and/or antiviral effect against viral pathogens that include, for example, viruses from the family Adenoviradae; Arenaviridae (for example, Ippy virus and Lassa virus); Birnaviridae; Bunyaviridae; Caliciviridae; Coronaviridae; Filoviridae; Flaviviridae (for example, yellow fever virus, dengue fever virus and hepatitis C virus); Hepadnaviradae (for example, hepatitis B virus); Herpesviradae (for example, human herpes simplex virus 1); Orthomyxoviridae (for example, influenza virus A, B and C); Paramyxoviridae (for example, mumps virus, measles virus and respiratory syncytial virus); Picornaviridae (for example, poliovirus and hepatitis A virus); Poxviridae; Reoviridae; Retroviradae (for example, BLV-HTLV retrovirus, HIV-1, HIV-2, bovine immunodeficiency virus and feline immunodeficiency virus); Rhabodoviridae (for example, rabies virus), and Togaviridae (for example, rubella virus). Non-limiting examples of relevant pathogenic viruses include, but are not limited to, various strains of the influenza virus, cytomegalovirus, various strains of respiratory syncytial virus (including human respiratory syncytial virus and specific animal strains), various strains of parainfluenza virus (including human parainfluenza virus and specific animal strains), coronavirus (including human coronavirus, SARS coronavirus, MERS coronavirus, and Covid-19 coronavirus), rhinovirus (including human rhinovirus), enterovirus (including human enterovirus), adenovirus (including human adenovirus), bocavirus (including human bocavirus), metapneumovirus (including human metapneumovirus), dengue virus, various hepatitis viruses, human immunodeficiency virus (HIV), West Nile virus, rabies virus, human papilloma virus (HPV), Epstein Barr virus (EBV) and polyoma virus. In certain embodiments of the invention, the GO-Ag⁺ nanocomposites exhibit virucidal and/or antiviral effect against influenza virus, a flavivirus (such as dengue fever virus or yellow fever virus), a parainfluenza virus, human metapneumovirus, respiratory syncytial virus, coronavirus (such as Covid-19 coronavirus, SARS coronavirus, MERS coronavirus), a rhinovirus or an adenovirus.

In further embodiments, the GO-Ag⁺ nanocomposites described herein, and/or compositions or formulations comprising these nanocomposites, exhibit antibacterial effect against bacterial pathogens. According to certain embodiments, bacterial pathogens include gram positive bacteria. In other embodiments, the bacterial pathogens include gram negative bacteria. Bacterial pathogens include, for example, various species of the Bacillus, Yersinia, Franscisella, Haemophilus, Streptococcus, Staphylococcus, Pseudomonas, Mycobacterium, and Burkholderia genus of bacteria. In certain embodiments, the GO-Ag⁺ nanocomposite compositions exhibit antibacterial effect against respiratory bacterial pathogens. Non-limiting examples of relevant pathogenic bacterial species include, but are not limited to, Bacillus anthracis, Yersinia pestis, Francisella tularensis, Streptococcus pnemoniae, Staphylococcus aureus, Pseudomonas aeruginosa, Burkholderia cepacia, Corynebacterium diphtheriae, Legionella pneumophila, Mycoplasma pneumoniae, Chlamydophila pneumoniae, Mycobacterium tuberculosis, Moraxella catarrhalis, Haemophilus influenzae, Klebsiella pneumoniae, Escherichia coli, Coxiella burnetii, Clostridia spp. and Shigella spp.

In certain embodiments, the GO-Ag⁺ nanocomposites described herein, and/or compositions or formulations comprising these nanocomposites, exhibit antifungal effect against fungal pathogens. Fungal pathogens include, for example, Histoplasma capsulatum, Coccidiodes immitis, Blastomyces dermatitidis, Cryptococcus neoformans, Aspergillus fumigatus, Candida albicans and Pneumocystis carinii.

In further embodiments, the GO-Ag⁺ nanocomposites described herein exhibit an antimicrobial efficacy against antimicrobial resistant (AMR) and/or multidrug resistant (MDR) microbial pathogens. In certain embodiments, the AMR and/or MDR pathogens include Pseudomonas aeruginosa, Escherichia coli, Klebsiella pneumoniae, Serratia marcescens, Acinetobacter baumanii, Stenotrophomonas maltophilia, Streptococcus pneumonia, Staphylococcus aureus, Candida auris, influenza virus, Extended Spectrum Beta-lactamase (ESBL) Escherichia coli, ESBL Klebsiella pneumoniae, Carbapenem Resistant Organisms (CRO) Enterobacter spp., Penicillin-resistant Streptococcus pneumonia, CA-MRSA, HA-MRSA, and Acinetobacter baumanii complex. In other embodiments, the GO-Ag⁺ nanocomposites described herein exhibit an antimicrobial efficacy against antimicrobial resistant (AMR) and/or multidrug resistant (MDR) microbial pathogens known as the ESKAPE pathogens. According to such embodiments, the GO-Ag⁺ nanocomposites described herein exhibit an antimicrobial efficacy against any one or more of Enterococcus faecium, Staphylococcus aureus, Klebsiella pneumoniae, Acinetobacter baumannii, Pseudomonas aeruginosa, and Enterobacter spp.

### Evaluation of Efficacy

The efficacy of the GO-Ag⁺ nanocomposites described herein, and/or compositions or formulations comprising these nanocomposites, in producing a therapeutic effect can be evaluated by standard techniques known in the art. For example, for therapeutic studies, standard animal models of infection can be employed with the animals being treated with the GO-Ag⁺ nanocomposite compositions at an appropriate time post-infection. Such studies involve the administration of the GO-Ag⁺ nanocomposite compositions to groups of test animals (such as mice) by standard techniques at an appropriate time post-infection with a pathogen. Control groups comprising untreated animals and/or animals treated with a known antimicrobial, or other positive control, are set up in parallel. The animals are monitored for development of conditions associated with infection including, for example, body temperature, weight, and the like. In certain cases, for example when the pathogen is associated with mortality, survival is also a suitable marker. The extent of infection can also be assessed, if desired, by measurement of microbial titers using standard techniques after sacrifice of the animal.

Other standard techniques may also be employed to assess the compositions, including, for example, evaluation of efficacy in combination with conventional prophylactic or therapeutic drugs in various animal models of infection and disease known in the art.

### Pharmaceutical Compositions and Administration

The present invention provides for pharmaceutical compositions comprising the GO-Ag⁺ nanocomposite and one or more pharmaceutically acceptable carriers, diluents and/or excipients. If desired, other active ingredients may be included in the compositions, for example, additional immune stimulating compounds, standard therapeutics, or the like.

The pharmaceutical compositions can be formulated for administration by a variety of routes. For example, the compositions can be formulated for oral, topical, rectal, nasal, ocular, otic, parenteral administration, or for administration by inhalation or spray. The term parenteral as used herein includes subcutaneous injections, intravenous, intramuscular, intrathecal, intrasternal injection or infusion techniques. Intranasal administration to the subject includes administering the composition to the mucous membranes of the nasal passage or nasal cavity of the subject.

In some embodiments, the pharmaceutical compositions are formulated for topical and/or mucosal administration (the terms topical and mucosal are used interchangeably herein). Topical or mucosal administration may include, for example, oral, ocular, otic, intranasal, aerosol, rectal or vaginal administration. The preparations for topical or mucosal administration include transdermal devices, aerosols, creams, lotions or powders pending on the topical or mucosal site. In certain embodiments, the pharmaceutical compositions are formulated for intranasal or pulmonary administration. In some embodiments, the pharmaceutical compositions are formulated for rectal or vaginal administration.

The pharmaceutical compositions comprise an effective amount of the GO-Ag⁺ nanocomposite. The effective amount for a given indication can be estimated initially, for example, in animal models, usually in rodents, rabbits, dogs, pigs or primates. The animal model may also be used to determine the appropriate concentration range and route of administration. Such information can then be used to determine useful doses and routes for administration in the animal to be treated, including humans. It is contemplated that one or more doses may be used to treat the subject, and these may be administered on the same day or over the course of several days or weeks.

Compositions formulated as aqueous suspensions may contain the GO-Ag⁺ nanocomposite in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth, gum acacia, hydrogels, carbomer, alginate, polyacrylic acid, and polyethylene glycol; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

In certain embodiments, the pharmaceutical compositions may be formulated as oily suspensions by suspending the GO-Ag⁺ nanocomposite in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. These compositions can be preserved by the addition of an anti-oxidant such as ascorbic acid.

In certain embodiments, the pharmaceutical compositions may be formulated as a dispersible powder or granules, which can subsequently be used to prepare an aqueous suspension by the addition of water. Such dispersible powders or granules provide the GO-Ag⁺ nanocomposite in admixture with one or more dispersing or wetting agents, suspending agents and/or preservatives. Suitable dispersing or wetting agents and suspending agents are exemplified by those already mentioned above. Additional excipients, for example, colouring agents, can also be included in these compositions.

Pharmaceutical compositions of the invention may also be formulated as oil-in-water emulsions in some embodiments. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate.

In certain embodiments, the pharmaceutical compositions may be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the pharmaceutical compositions may contain preservatives such as antimicrobial agents, antioxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum or skimmed milk) together with a suitable buffer (e.g. phosphate buffer). The pH and exact concentration of the various components of the composition may be adjusted according to well-known parameters.

Sterile compositions can be prepared for example by incorporating the GO-Ag⁺ nanocomposite in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some exemplary methods of preparation are vacuum-drying and freeze-drying techniques which yield a powder of the active ingredient plus any additional desired ingredient from a previously sterile-filtered solution thereof.

Contemplated for use in certain embodiments of the invention are various mechanical devices designed for pulmonary or intranasal delivery of therapeutic products, including but not limited to, nebulizers, metered dose inhalers, powder inhalers and nasal spray devices, all of which are familiar to those skilled in the art.

Metered dose inhalers typically use a propellant gas and require actuation during inspiration. Dry powder inhalers use breath-actuation of a mixed powder. Nebulizers produce aerosols from solutions, while metered dose inhalers, dry powder inhalers, and the like generate small particle aerosols.

Some specific examples of commercially available devices suitable for the practice of this invention are the ULTRA VENT® nebulizer (Mallinckrodt, Inc., St. Louis, Mo.), the ACORN II® nebulizer (Marquest Medical Products, Englewood, Colo.), the MISTY-NEB® nebulizer (Allegiance, McGraw Park, Ill.), the AEROECLIPSE® nebulizer (Trudell Medical International, Canada), the Accuspray™ nasal spray device (Becton Dickinson), the Mucosal Atomization Device (MAD300) (Wolfe Tory Medical), the OptiNose device (OptiNose, Oslo, Norway), the Nektar DPI system (Nektar Therapeutics, Inc., San Carlos, Calif.), the AERx pulmonary drug delivery system (Aradigm Corporation, Hayward, Calif.), the Spiros® device (Dura Pharmaceuticals), and the Respimat® device (Boehringer Ingelheim).

All such devices require the use of formulations suitable for the dispensing of the GO-Ag⁺ nanocomposite. Typically, each formulation is specific to the type of device employed and may involve the use of an appropriate propellant material, in addition to the usual diluents, adjuvants and/or carriers useful in therapy as would be understood by a worker skilled in the art. Also, the use of liposomes, microcapsules or microspheres, inclusion complexes, or other types of carriers is contemplated.

Thus, in some embodiments, the invention provides for pharmaceutical compositions that are formulated for delivery via an intranasal or pulmonary route in, for example, lyophilized powder form, in an aerosolized liquid form, or in a gel form. These routes of administration can also allow for easy administration in the event of the need for mass distribution.

Formulations suitable for use with a nebulizer, either jet or ultrasonic, will typically comprise the GO-Ag⁺ nanocomposite in an aqueous medium at a suitable concentration, for example, about 0.01 μg to 25 mg, or about 0.01 μg to 0.1 mg, or about 0.02 μg to 0.1 mg, or about 0.1 mg to 10 mg, of GO-Ag⁺ nanocomposite per mL of solution.

The formulation may also include a buffer and a simple sugar (for example, for regulation of osmotic pressure), and/or human serum albumin ranging in concentration from about 0.1 to about 10 mg/ml. Examples of buffers that may be used include, but are not limited to, sodium acetate, citrate and glycine. Typically, the buffer will have a composition and molarity suitable to adjust the solution to a pH in the range of 3 to 9. Generally, buffer molarities of from 1 mM to 50 mM are suitable for this purpose. Examples of excipients, usually in amounts ranging from about 1% to about 90% by weight (for example, from about 1% to about 50% by weight, or about 5% to about 30% by weight) of the formulation include, but are not limited to, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; alditols, such as mannitol, xylitol, xylose, maltitol, lactitol, xylitol sorbitol (glucitol), sorbitose, pyranosyl sorbitol, myoinositol and the like; and glycine, CaCl₂), hydroxyectoine, ectoine, gelatin, di-myo-inositol phosphate (DIP), cyclic 2,3-diphosphoglycerate (cDPG), 1,1-di-glycerol phosphate (DGP), β-mannosylglycerate (firoin), β-mannosylglyceramide (firoin A), proline betaine and/or derivatives, as well as combinations thereof.

The nebulizer formulation may also contain a surfactant to reduce or prevent surface induced aggregation of the composition components caused by atomization of the solution in forming the aerosol. Various conventional surfactants can be employed, such as polyoxyethylene fatty acid esters and alcohols, and polyoxyethylene sorbitan fatty acid esters. Amounts will generally range between about 0.001% and about 4% by weight of the formulation. A non-limiting example of a surfactant for this purpose is polyoxyethylene sorbitan monooleate.

In certain embodiments, the pharmaceutical compositions can be delivered in powder form using, for example, a metered dose inhaler device. This powder may be produced by lyophilization and may also contain a stabilizer such as human serum albumin (HSA). Additionally, one or more of the following may be added as an excipient to the composition, if necessary, to enhance one or more features (for example, to facilitate dispersal of the powder from a device, to increase the shelf-life of the composition, or to improve the stability of the composition during lyophilization): monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; alditols, such as mannitol, xylitol, xylose, maltitol, lactitol, xylitol sorbitol (glucitol), sorbitose, pyranosyl sorbitol, myoinositol and the like; and glycine, CaCl₂), hydroxyectoine, ectoine, gelatin, di-myo-inositol phosphate (DIP), cyclic 2,3-diphosphoglycerate (cDPG), 1,1-di-glycerol phosphate (DGP), β-mannosylglycerate (firoin), β-mannosylglyceramide (firoin A), proline, betaine and/or derivatives as well as combinations thereof. The amount added to the composition can range from about 0.01% to 200% (w/w), for example, from about 1% to 50% (w/w), or from about 5% to 30% (w/w) of the GO-Ag⁺ nanocomposite present. Such formulations are then lyophilized and milled to the desired particle size. Typically, the particles of the powder have a median diameter less than about 50 μm, for example, between about 1.5 μm and 10 μm. The mean particle diameter can be measured using conventional equipment, such as a Cascade Impactor (Andersen, Ga.).

The powder may be suspended in a propellant with the aid of a surfactant. The propellant may be one of a variety of conventional materials employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

In certain embodiments of the invention, the pharmaceutical compositions are administered intranasally and the compositions are therefore formulated as nasal gels, creams, pastes or ointments that provide a more sustained contact with the nasal mucosal surfaces. These formulations typically have a viscosity between about 10 and about 250,000 centipoise (cps), for example, between about 2500 about 100,000 cps, or between about 5,000 and 50,000 cps. Such formulations may be based upon, for example, alkylcelluloses and/or other biocompatible carriers of high viscosity well known to the art. A non-limiting example of an alkylcellulose is methylcellulose, which can be included in a suitable concentration, for example, between about 5 mg and about 1000 mg per 100 ml of carrier, or between about 25 mg and about 750 mg per 100 ml of carrier. In certain embodiments, the carrier containing the GO-Ag⁺ nanocomposite may be soaked into a suitable substrate, for example a fabric material, such as gauze, that can be applied to the nasal mucosal surfaces to allow for penetration of the GO-Ag⁺ nanocomposite into the mucosa.

In certain embodiments, gel formulations may also include a permeation enhancer (penetration enhancer). Permeation enhancers include, but are not limited to, sulfoxides such as dimethylsulfoxide and decylmethylsulfoxide; surfactants such as sodium laurate, sodium lauryl sulfate, cetyltrimethylammonium bromide, benzalkonium chloride, poloxamer (231, 182, 184), Tween (20, 40, 60, 80) and lecithin; the 1-substituted azacycloheptan-2-ones, particularly 1-n-dodecylcyclazacycloheptan-2-one; fatty alcohols such as lauryl alcohol, myristyl alcohol, oleyl alcohol and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate, amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine, terpenes; alkanones, and organic acids, particularly salicylic acid and salicylates, citric acid and succinic acid. The permeation enhancer may be present in an amount from about 0.1% to about 30% w/w. The gel compositions may also include a buffering agent, for example, carbonate buffers, citrate buffers, phosphate buffers, acetate buffers, hydrochloric acid, lactic acid, tartaric acid, inorganic and organic bases. The buffering agent may be present in a concentration of about 1 to about 10 weight percent, for example, about 2 to about 5 weight percent, depending on the type of buffering agent(s) used, as known by the one skilled in the art. Concentrations of the buffering agent(s) may vary, however, and in some embodiments the buffering agent may replace up to 100% of the water amount within the composition.

In certain embodiments of the invention, the pharmaceutical compositions are formulated for rectal or vaginal administration and may be presented as a suppository, which may be prepared by mixing the active ingredient(s) with one or more suitable non-irritating excipients or carriers. Non-limiting examples of excipients or carriers include cocoa butter, polyethylene glycol, a suppository wax or salicylate and which is solid at room temperature, but liquid at body temperature and, therefore, will melt in the rectum or vaginal cavity and release the active ingredient(s). Formulations of the present invention which are suitable for vaginal administration also include pessaries, tampons, creams, gels, pastes, foams or spray formulations containing such carriers as are known in the art to be appropriate.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Methods and Uses

The present invention provides for the use of GO-Ag⁺ nanocomposite compositions for preventing or treating at least one microbial infection in a subject. In certain embodiments, the at least one microbial infection is a respiratory tract infection in a subject. The subject may be a human or a non-human animal. The compositions are useful, for example, in the treatment or prevention of infection, including chronic infection.

In certain embodiments, GO-Ag⁺ nanocomposite compositions may be used to treat or prevent an infection, for example, an infection with a viral pathogen, a bacterial pathogen, and/or a fungal pathogen. In some embodiments, GO-Ag⁺ nanocomposite compositions may be used to treat or prevent an infection within the mucosa and/or in the respiratory system. In some embodiments, GO-Ag⁺ nanocomposite compositions may be used to treat or prevent infection with an antimicrobial resistant and/or multidrug resistant microbial pathogen. According to some embodiments, the GO-Ag⁺ nanocomposite compositions are administered topically for the treatment of infections on the skin, for example, in the form of creams, ointments, and bandages infused with the GO-Ag⁺ nanocomposite compositions. In accordance with certain embodiments of the invention, the GO-Ag⁺ nanocomposite compositions are administered to elicit a prophylactic or therapeutic effect within the mucosa and/or in the respiratory system. Administration via intranasal or pulmonary routes, for example, can be used to provide treatment in the respiratory tract. Administration via vaginal routes, for example, can be used to treat vaginal infections. Other routes of administration are also contemplated.

In certain embodiments, GO-Ag⁺ nanocomposite compositions may be administered to treat or prevent infection with a bacterial pathogen. Bacterial pathogens include, for example, various species of the Bacillus, Yersinia, Franscisella, Haemophilus, Streptococcus, Staphylococcus, Pseudomonas, Mycobacterium, and Burkholderia genus of bacteria. In certain embodiments, GO-Ag⁺ nanocomposite compositions may be administered to treat or prevent respiratory infection with a bacterial pathogen. Non-limiting examples of relevant pathogenic bacterial species include, but are not limited to, Bacillus anthracis, Yersinia pestis, Francisella tularensis, Streptococcus pnemoniae, Staphylococcus aureus, Pseudomonas aeruginosa, Burkholderia cepacia, Corynebacterium diphtheriae, Legionella pneumophila, Mycoplasma pneumoniae, Chlamydophila pneumoniae, Mycobacterium tuberculosis, Moraxella catarrhalis, Haemophilus influenzae, Klebsiella pneumoniae, Escherichia coli, Coxiella burnetii, Clostridia spp. and Shigella spp. In certain embodiments of the invention, GO-Ag⁺ nanocomposite compositions may be administered to treat infection with a bacteria associated with bacterial pneumonia, for example, one or more of S. pneumoniae, S. aureus, H. influenzae, K. pneumoniae, P. aeruginosa, E. coli, M. catarrhalis, C. burnetii, M. pneumoniae, L. pneumoniae, C. pneumoniae and Y. pestis. In certain embodiments of the invention, GO-Ag⁺ nanocomposite compositions may be administered to treat a vaginal or intestinal bacterial pathogen, for example, Shigella spp., Salmonella spp., E. coli or Chlamydia trachomatis.

In certain embodiments, GO-Ag⁺ nanocomposite compositions may be administered to treat or prevent infection with a viral pathogen. Viral pathogens include, for example, viruses from the family Adenoviradae; Arenaviridae (for example, Ippy virus and Lassa virus); Birnaviridae; Bunyaviridae; Caliciviridae; Coronaviridae; Filoviridae; Flaviviridae (for example, yellow fever virus, dengue fever virus and hepatitis C virus); Hepadnaviradae (for example, hepatitis B virus); Herpesviradae (for example, human herpes simplex virus 1); Orthomyxoviridae (for example, influenza virus A, B and C); Paramyxoviridae (for example, mumps virus, measles virus and respiratory syncytial virus); Picornaviridae (for example, poliovirus and hepatitis A virus); Poxviridae; Reoviridae; Retroviradae (for example, BLV-HTLV retrovirus, HIV-1, HIV-2, bovine immunodeficiency virus and feline immunodeficiency virus); Rhabodoviridae (for example, rabies virus), and Togaviridae (for example, rubella virus). Non-limiting examples of relevant pathogenic viruses include, but are not limited to, various strains of the influenza virus, cytomegalovirus, various strains of respiratory syncytial virus (including human respiratory syncytial virus and specific animal strains), various strains of parainfluenza virus (including human parainfluenza virus and specific animal strains), coronavirus (including human coronavirus, SARS coronavirus, MERS coronavirus, and Covid-19 coronavirus), rhinovirus (including human rhinovirus), enterovirus (including human enterovirus), adenovirus (including human adenovirus), bocavirus (including human bocavirus), metapneumovirus (including human metapneumovirus), dengue virus, various hepatitis viruses, human immunodeficiency virus (HIV), West Nile virus, rabies virus, human papilloma virus (HPV), Epstein Barr virus (EBV) and polyoma virus. In certain embodiments of the invention, GO-Ag⁺ nanocomposite compositions may be administered to treat or prevent infection with an influenza virus, a flavivirus (such as dengue fever virus or yellow fever virus), a parainfluenza virus, human metapneumovirus, respiratory syncytial virus, coronavirus (such as Covid-19 coronavirus, SARS coronavirus, MERS coronavirus), a rhinovirus or an adenovirus.

In certain embodiments, GO-Ag⁺ nanocomposite compositions may be administered to treat or prevent infection with a fungal pathogen. Fungal pathogens include, for example, Histoplasma capsulatum, Coccidiodes immitis, Blastomyces dermatitidis, Cryptococcus neoformans, Aspergillus fumigatus, Candida albicans and Pneumocystis carinii.

In further embodiments, GO-Ag⁺ nanocomposite compositions may be administered to treat or prevent infection with an antimicrobial resistant (AMR) and/or multidrug resistant (MDR) microbial pathogen. AMR and/or MDR pathogens include, for example, Pseudomonas aeruginosa, Escherichia coli, Klebsiella pneumoniae, Serratia marcescens, Acinetobacter baumanii, Stenotrophomonas maltophilia, Streptococcus pneumonia, Staphylococcus aureus, Candida auris, influenza virus, Extended Spectrum Beta-lactamase (ESBL) Escherichia coli, ESBL Klebsiella pneumoniae, Carbapenem Resistant Organisms (CRO) Enterobacter spp., Penicillin-resistant Streptococcus pneumonia, CA-MRSA, HA-MRSA, and Acinetobacter baumanii complex. In other embodiments, GO-Ag⁺ nanocomposites compositions may be administered to treat or prevent infection with an antimicrobial resistant (AMR) and/or multidrug resistant (MDR) microbial pathogen known as ESKAPE pathogen including, for example, Enterococcus faecium, Staphylococcus aureus, Klebsiella pneumoniae, Acinetobacter baumannii, Pseudomonas aeruginosa, and Enterobacter spp.

### Kits

The present invention additionally provides for kits comprising GO-Ag⁺ nanocomposite compositions. In certain embodiments the kit is portable and may be carried on a person. The kit may optionally further include a pathogen detector. The kit may also optionally contain a gas or mechanical propellant for the GO-Ag⁺ nanocomposite compositions.

Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the GO-Ag⁺ nanocomposite composition.

The components of the kits may be packaged as solutions, or in powdered or lyophilized form. When components of the kit are provided in dried or lyophilised form, the kit can additionally contain a suitable solvent for reconstitution of the dried or lyophilised components. Irrespective of the number or type of containers, the kits of the invention also may comprise an instrument for assisting with the administration of the composition to a patient. Such an instrument may be an inhaler, nebulizer, nasal spray device, syringe, pipette, or similar medically approved delivery vehicle. In certain embodiments, the container comprising the composition may itself be such an instrument.

To gain a better understanding of the invention described herein, the following examples are set forth. It will be understood that these examples are intended to describe illustrative embodiments of the invention and are not intended to limit the scope of the invention in any way.

### EXAMPLES

### Example 1: Preparation of Graphene—Silver Cation Nanocomposite

GO-Ag⁺ nancomposite was prepared according to the following method based on 1 g graphite powder.

### Synthesis of GO

1 g graphite powder was soaked in 30 mL H₂SO₄ (98%) solution under the fume hood in an Erlenmeyer under stirring at 50° C. and 300 rpm for 18 h. The suspension was sonicated 3 times every 6 h using a bath sonicator for 30 min. Then, 4 g KMnO₄ was added gradually to the previous mixture. An ice/water bath was used to decrease the temperature of the mixture during the exothermic oxidation reactions. The mixture was kept under continuous stirring for up to 12 h at 35° C. Bath sonication (15-30 min) was used 3 times after 8 h of stirring in this stage. Next, 100 mL of cold distilled water (2-5° C.) was added to the previous mixture. In this stage, the temperature of the mixture was kept below 5° C. using an ice-bath. Then, H₂O₂ (30%) was added drop-by-drop to the diluted mixture until the color of the mixture changed from dark brown to yellow. Before purification, 30 min bath sonication of the mixture was used to accelerate the separation of exfoliated GO nanosheets from each other. The final suspension was washed 3 times with HCl (1 M) and three times with a mixture of water/ethanol (8:2 v/v). Bath sonication (30 min) was used between the purification steps to accelerate the removal of impurities intercalated between the GO layers. pH of the sample was adjusted between 3-4 during the centrifuging by 1 M KOH solution for better sedimentation. The final GO nanosheets were dispersed in distilled water and stored for the next usages.

### Silver (Ag⁺) Doped GO Synthesis

Graphene oxide (GO) nanosheets as the platform for cationic silver were synthesized. To synthesize Ag doped GO nanosheets, 1 g dried pristine GO powder was dispersed in 100 mL DI water in a 250 mL Erlenmeyer flask using a bath sonicator for 30 min. The pH of the GO suspension was adjusted at 10 using NaOH solution (0.1 M). Then, 2 mL of the AgNO₃ solutions (10 M) was added to the previous suspension under stirring (400 rpm). The mixture was stirred for 20 h at 60° C. Finally, Ag doped GO nanosheets were collected using a centrifuge (4000 rpm), washed with DI water 3 times, and dried in an oven at ˜60° C. overnight.

### Example 2: Complex Bonding of Cationic Silver to go Nanosheets

GO surfaces are known to act like reducing agents. It is possible for some of the Ag⁺ cation to be reduced to metallic Ag(0). This reduction of the cation Ag⁺ could also be associated with simultaneous oxidation of other graphene functionalities (like quinone to hydroquinone). It has also been observed that species with C and O (like phenolic, carbonyl) would oxidise during the process. On the other hand, Ag⁺ could be on the surface in +1 oxidation state or simply coordinated with the oxygen or other functional groups on the GO surface. Where Ag(0) particles are present, they can contact the microorganism (pathogen) and directly act on them by either interfering with the DNA/RNA replication or denaturing other proteins in the cell (ribosome, cell membrane, etc.); alternatively, Ag would come in contact with liquid (surrounding the pathogen), thus oxidizing Ag(0) to Ag (+) and the latter disrupts the cell membrane or denatures the proteins.

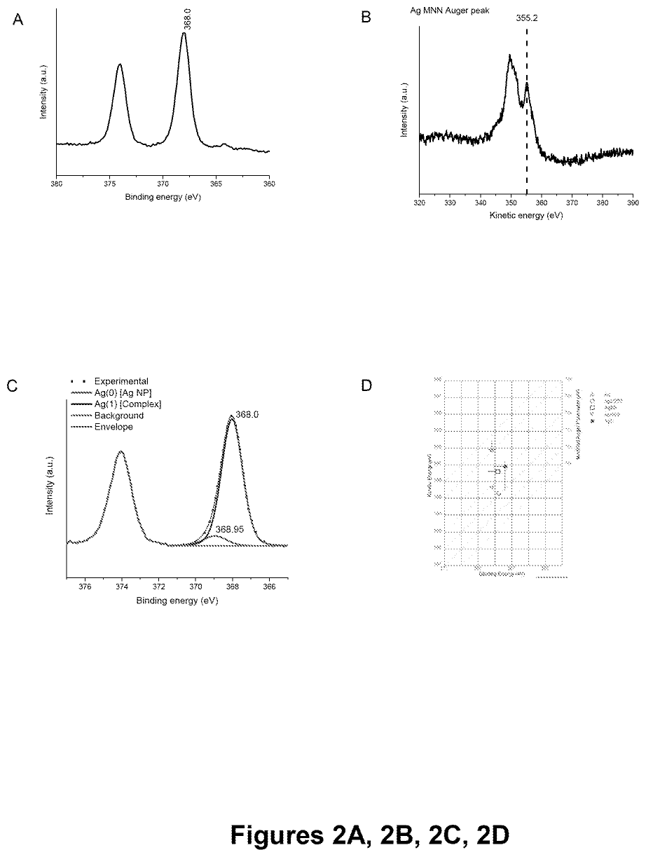

To study the silver species attached to the GO-Ag⁺ nanocomposite, and to consider the inclusion of Ag(1)-complexes as well as (Ag(0)) nanoparticles on the GO, peak fitting was performed. X-ray photoelectron spectroscopy (XPS) and Auger Electron Spectroscopy (AES) of one exemplary product according to the present invention (defined in Table 1) was analyzed (see FIGS. 2A, 2B, Table 1).

TABLE 1
Identified Product Components

| Component | Peak, eV | atm % | wt % |
|---|---|---|---|
| C 1s | 284.82 | 71.07 | 49.41 |
| O 1s | 532.42 | 24.46 | 22.65 |
| Ag 3d | 368.02 | 4.47 | 27.94 |

TABLE 2
Distribution of Ag1-Complex and Clustered Ag(0)

| Component | Peak, eV | FWHM | Composition % |
|---|---|---|---|
| Ag(0) [Ag NP cluster] | 368.95 | 1.5 | 7.98 |
| Ag(1) [Ag(1) | 368.02 | 1.34 | 92.02 |
| Complex/Ag2CO3] | | | |

TABLE 3
Identified Product Components

| | | | | | C/O, |
|---|---|---|---|---|---|
| Element | Position | FWHM | At % conc | Wt % conc | a/a |
| O 1s | 532.54 | 1.92 | 26.54 | 32.1 | 2.74 |
| C 1s | 286.74 | 3.48 | 72.67 | 65.98 | |
| S 2p | 168.84 | 2.2 | 0.79 | 1.92 | |

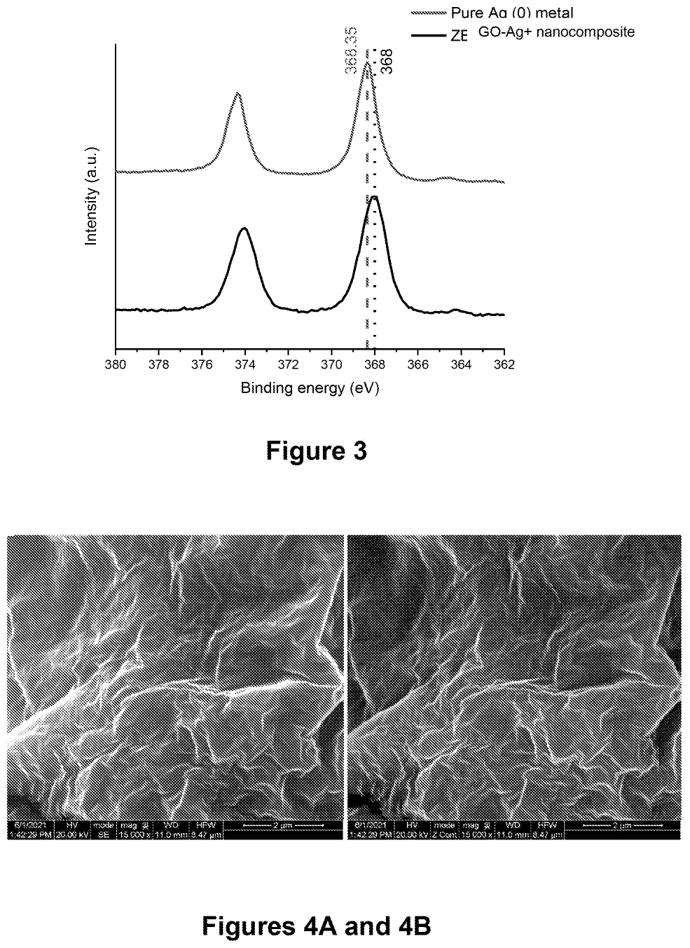

The calculated Auger parameter values for the exemplary product was 723.2 eV, confirming the significant composition is not Ag or Ag2O (see FIGS. 2C, 2D). Peak fitting of the Ag 3d5/2 data (Table 2) indicates that approximately 92% of the silver in the exemplary product is in a Ag(1)-complex form and the remaining 8% could be attributed to clustered Ag(0)-nanoparticle forms. No form of nitrogen was detected, thus confirming the removal of nitrates in the final product. The downward shift of the Ag3d5/2 peak of the exemplary product when compared to Ag(0) clearly indicated the chemical state of the former to be Ag(1) (FIG. 3). Elemental C and O composition of the exemplary product remained similar to the GO elemental composition, except for the addition of approximately 4.5 at % Ag (Tables 1 and 3). This suggests that the Ag(1) ions are complexing with the existing functional groups on the GO. Positively charged Ag(1) ions, which were initially introduced into the system via the addition of AgNO₃, are electrostatically attracted towards the negatively charged functional groups with lone pair electrons on the GO surface to form complex bonds such as coordinate covalent bonds (both electrons shared in the bonding are from the functional group on the GO sheet which is the ligand in this case).

### Example 3: Morphological Characterization of Graphene-Silver Cation Nanocomposite

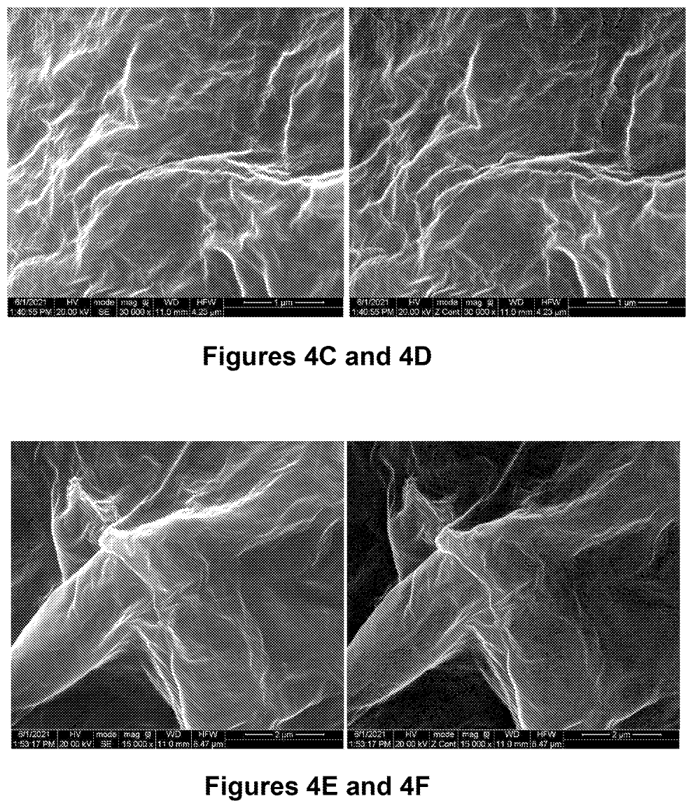

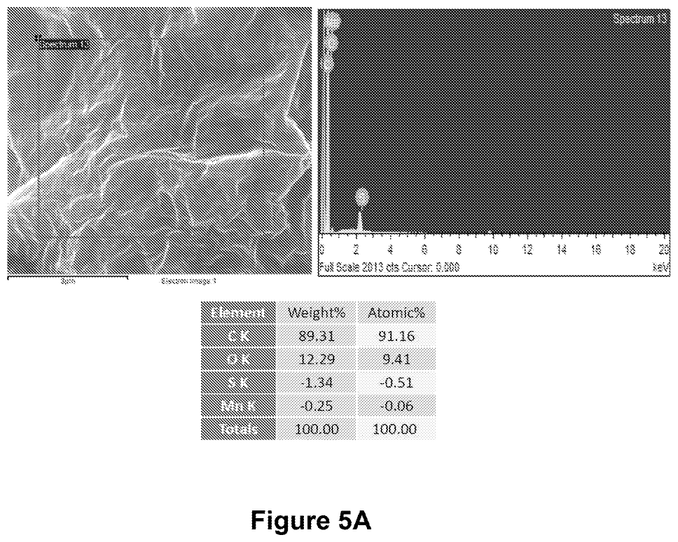

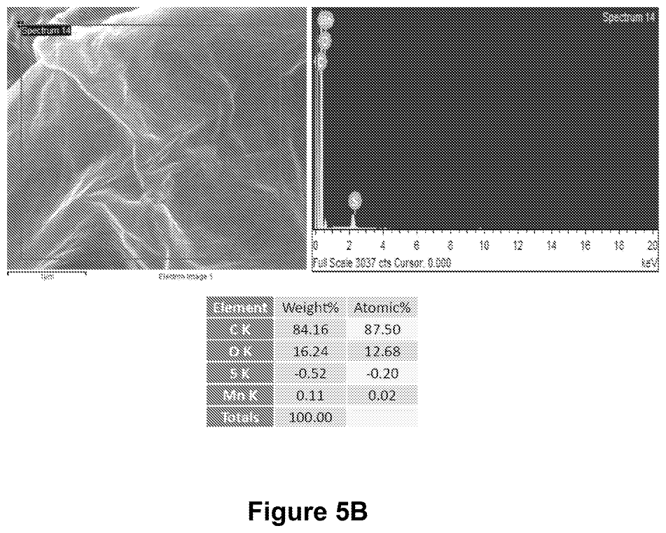

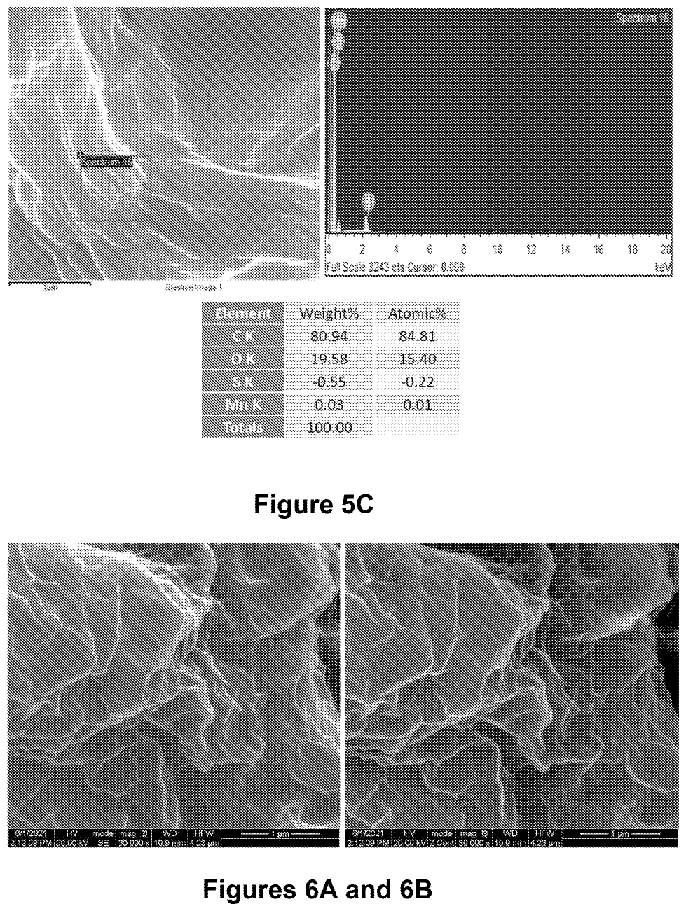

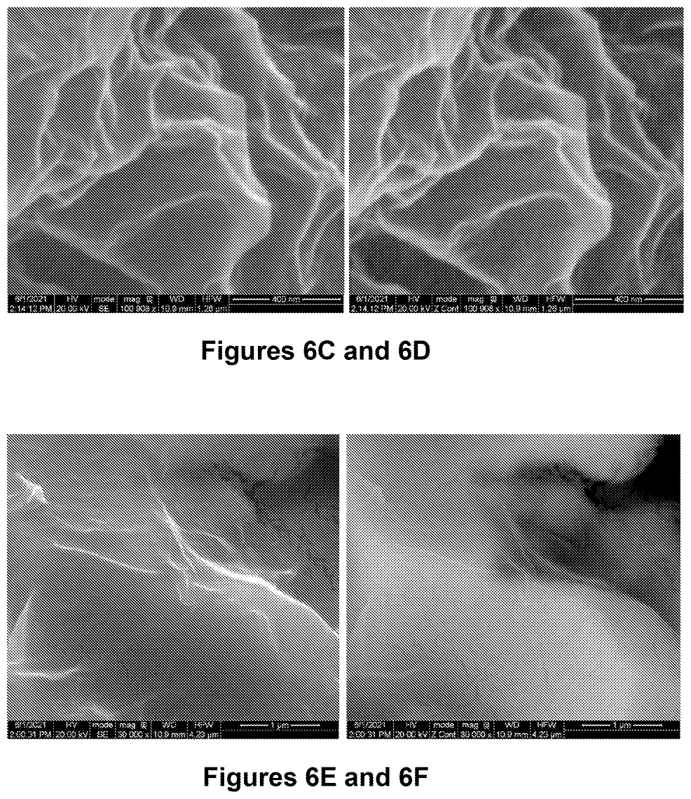

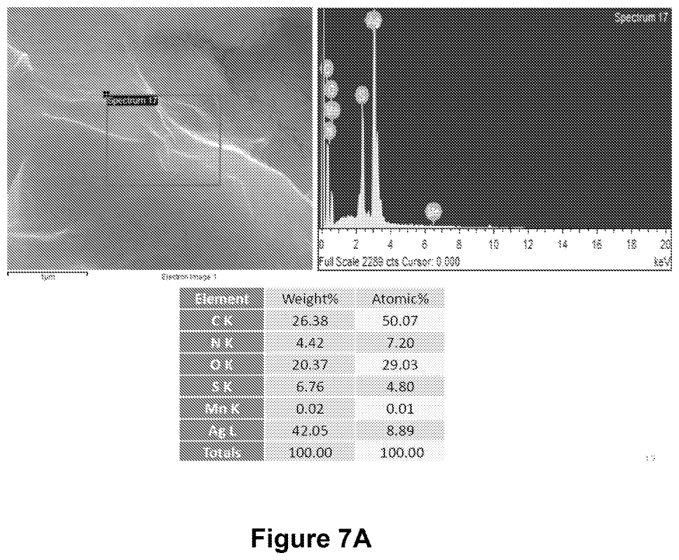

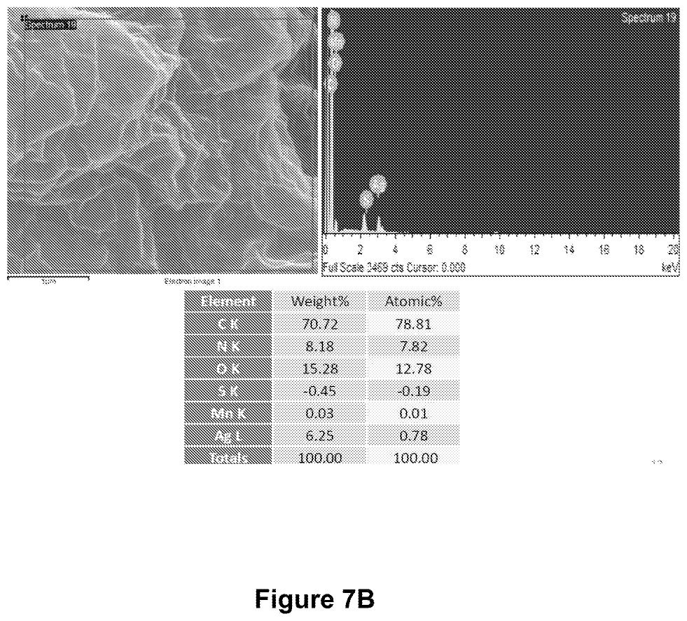

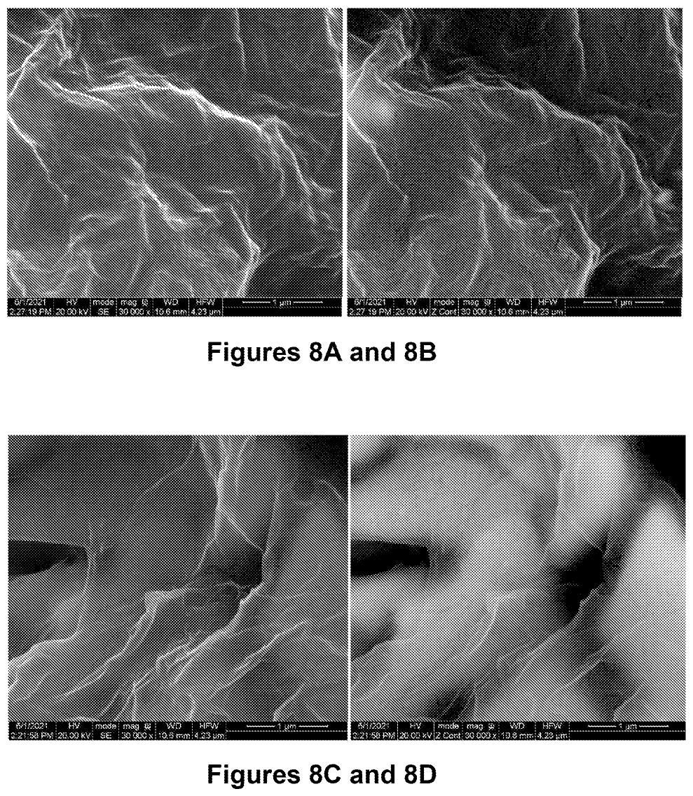

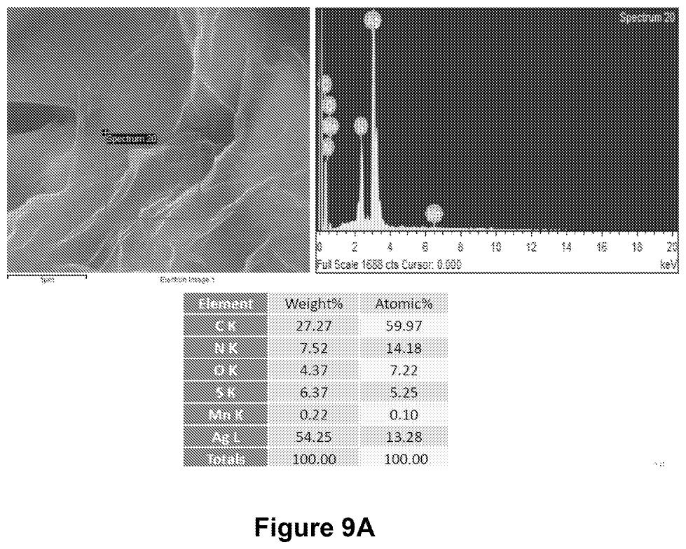

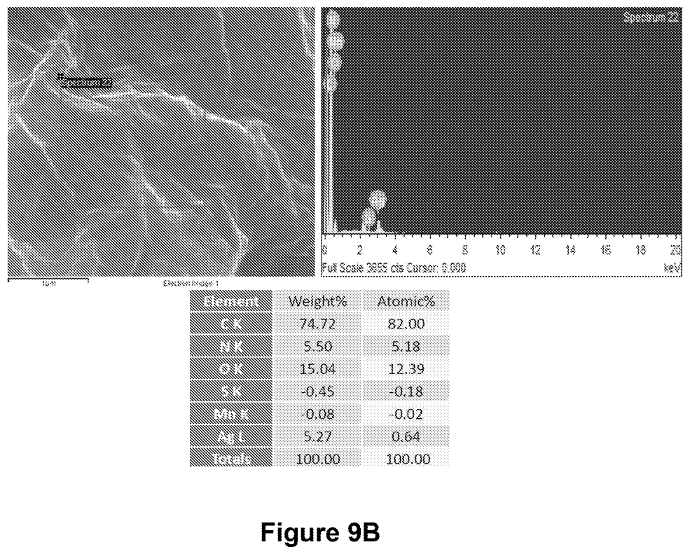

The surface morphology of GO-Ag⁺ nanocomposite was studied by scanning electron microscopy (SEM) and energy-dispersive X-ray spectroscopy (EDS) conducted on surfaces of GO samples, (a) before contact with the silver cations, (b) after contact but before purification (purification being removal of nitrates after supply of Ag⁺ from silver nitrate), and (c) after purification. The SEM analysis was done in two different modes of SE (scanning electron) and Backscattered-Electron (BSE or Z count); in the second mode, different components with different electron scattering level make different contrasts and provide more surface details (for example, the areas with more silver are brighter). Measurements were conducted on samples diluted in ethanol. Note that negative results should be read as zero. FIGS. 4A to 4F are SEM (SE) and SEM (Z count) images of GO before contact with the silver cation, and FIGS. 5A to 5C are EDS images and results charts for GO before contact with the silver cation. FIGS. 6A to 6F are SEM (SE) and SEM (Z count) images of GO after contact with the silver cation but before purification, and FIGS. 7A and 7B are EDS images and results charts for GO after contact with the silver cation but before purification. FIGS. 8A to 8D are SEM (SE) and SEM (Z count) images of GO after contact with the silver cation and after purification, and FIGS. 9A and 9B are EDS images and results charts for GO after contact with the silver cation and after purification.

### Example 4: Broad-Spectrum Antimicrobial Activity of Graphene-Silver Cation Nanocomposite

A macrobroth dilution method was used to determine the MIC of GO-Ag⁺ nanocomposite against 7 exemplary bacteria and 1 yeast following the Clinical Laboratory Standards Institute (CLSI) M7, M11, and M60 documents. The following organisms were tested:
1. Streptococcus pneumoniae (ATCC® 33400)
2. Haemophilus influenzae (ATCC® 51907D-5)
3. Streptococcus pyogenes (Group A Streptococcus) (ATCC® 12344D-5)
4. Moraxella catarrhalis (ATCC® 19606D-5)
5. Staphylococcus aureus (ATCC® 12600)
6. E. coli (ATCC® 10798)
7. Fusobacterium micleatum (ATCC® 25586D-5)
8. Candida albicans (ATCC® 14053)

GO-Ag⁺ nanocomposite (Sample #: 09-002-501; Oct. 22, 2020) was initially dissolved in sterile water to a concentration of 2 mg/mL. For S. pneumoniae, S. pyogenes, S. aureus, M. catarrhalis and E. coli, Mueller Hinton Broth was used. For H. influenzae, Haemophilus Test Broth was used and for Fusobacterium nucleatum, Fastidious Anaerobic Broth was used. The range of concentrations (ug/mL) tested were: 1, 0.5, 0.25, 0.125, 0.0625, 0.031, 0.0156, 0.008 and 0.004 (total of 9 target concentrations). Five replicates at each concentration were tested.

Each organism was prepared to a 0.5 McFarland standard (equivalent to 105 cfu/mL) and inoculated into each tube containing the decreasing concentrations of GO-Ag⁺ nanocomposite and incubated at 37° C. in 5% CO₂ for 18 to 24 hours, with the exception of the Fusobacterium nucleatum which was incubated anaerobically. Following incubation, the tubes were examined for turbidity. The lowest concentration showing no turbidity in all 5 replicates was considered as the MIC. A growth control (containing no GO-Ag⁺ nanocomposite) was used for each organism and each set of tests. A sterility control and growth control were used for each organism to ensure there was no contamination and that growth occurred in the broth medium in the absence of GO-Ag⁺, respectively.

### Results

Table 4 presents results demonstrating the antimicrobial activity of the GO-Ag⁺ nanocomposite, reflected by the MICs for each of the organisms tested, namely, Streptococcus pyogenes (Group A Streptococcus) (ATCC® 12344D-5), Staphylococcus aureus (ATCC® 12600), Streptocococcus pneumoniae (ATCC® 33400), Moraxella catarrhalis (ATCC® 19606D-5), Haemophilus influenzae (ATCC® 51907D-5), E. coli (ATCC® 10798), Candida albicans (ATCC® 14053), and Fusobacterium nucleatum (ATCC® 25586D-5). The MICs are based on complete inhibition in all 5 replicates where the “X” represents the lowest concentration of the GO-Ag⁺ nanocomposite at which no visible turbidity was observed.

TABLE 4
Broad- Spectrum Antimicrobial Activity (MIC) of GO-Ag⁺ Nanocomposite

| | Minimum Inhibitory Concentration (MIC, μg/mL) | | | | | | | | | Growth |
|---|---|---|---|---|---|---|---|---|---|---|
| Organism | 1 | 0.5 | 0.25 | 0.125 | 0.0625 | 0.031 | 0.0156 | 0.008 | 0.004 | Control |
| Streptococcus | | | | | | | X | | | Growth |
| pyogenes | | | | | | | (0.0156) | | | |
| (Group A | | | | | | | | | | |
| Streptococcus) | | | | | | | | | | |
| Staphylococcus | | | | | | X | | | | Growth |
| aureus | | | | | | (0.031) | | | | |
| Streptococcus | | | | | | | X | | | |
| pneumoniae | | | | | | | (0.0156) | | | |
| Moraxella | | | | | | | | X | | Growth |
| catarrhalis | | | | | | | | (0.008) | | |
| Haemophilus | | | | | X | | | | | |
| influenzae | | | | | (0.0625) | | | | | |
| Escherichia | | | | | | | X | | | Growth |
| coli | | | | | | | (0.0156) | | | |
| Candida | | | | | X | | | | | Growth |
| albicans | | | | | (0.0625) | | | | | |
| Fusobacterium | Growth | | | | | | | | | |
| nucleatum | | | | | | | | | | |

### Discussion

The results of this evaluation indicate that the GO-Ag⁺ nanocomposite is capable of inhibiting common aerobic bacteria and yeast at very low concentrations. However, it did not appear to inhibit the anaerobic bacteria used in this evaluation (Fusobacterium nucleatum) over the range of concentrations tested. This, however, was due to the Agar used as a medium which negates the activity of Ag⁺.

GO-Ag⁺ nanocomposite appears to be active against both gram positive (e.g. Streptococci and Staphylococci) and gram negative (e.g. E. coli, H. influenzae, M. catarrhalis) bacteria as well as common yeast (e.g. Candida albicans) at extremely low concentrations.

The relatively low concentrations of GO-Ag⁺ nanocomposite required to achieve an antimicrobial effect are well below the concentration required of commonly used antibiotics to show a similar effect.

Overall, the results of this evaluation demonstrate that GO-Ag⁺ nanocomposite is a novel compound with broad spectrum antibacterial, antiviral, and antifungal activity at extremely low concentrations of 0.008-0.0625 μg/mL.

### Example 5: Antimicrobial Activity of Graphene-Silver Cation Nanocomposite Against Antimicrobial Resistant and Multidrug Resistant Pathogens

The efficacy of the GO-Ag⁺ nanocomposite was further tested against a broad range of exemplary, non-limiting, antimicrobial resistant (AMR) and multidrug resistant (MDR) organisms, that include:

### 1. Gram Negative Bacteria:

Pseudomonas aeruginosa (2 separate isolates)
E. coli (Extended Spectrum Beta-Lactamase producer [ESBL])
E. coli (Carbapenem Resistant [CRO])
Klebsiella pneumoniae (ESBL)
Klebsiella pneumoniae (CRO)
Enterobacter aerogenes
Stenotrophomonas maltophilia

### 2. Gram Positive Bacteria:

Hospital Acquired-Methicillin Resistant Staphylococcus aureus (HA-MRSA)
Community Acquired-Methicillin Resistant Staphylococcus aureus (CA-MRSA)
Vancomycin Resistant Enterococcus faecium (VRE)
Penicillin-resistant Streptococcus pneumoniae

The same methodology was used as described in Example 4 and the results are presented in Table 5. In particular, GO-Ag⁺ nanocomposite (Sample #: 09-002-501; Oct. 22, 2020) was initially dissolved in sterile water to a concentration of 2 mg/mL. Mueller Hinton (MH) Broth was used for all organisms. The range of concentrations (μg/mL) tested were: 1, 0.5, 0.25, 0.125, 0.0625, 0.031, 0.0156, 0.008 and 0.004 (total of 9 target concentrations). Five replicates at each concentration were tested.

Each organism was prepared to a 0.5 McFarland standard (equivalent to 10⁵ cfu/mL) and inoculated into each tube containing the decreasing concentrations of GO-Ag⁺ and incubated at 37° C. in 5% CO₂ for 18 to 24 hours. Following incubation, the tubes were examined for turbidity. The lowest concentration showing no turbidity in all 5 replicates was considered as the MIC. Sub-cultures were performed to confirm a 99.9% reduction in growth for each organism compared to the growth control. A growth control (containing no GO-Ag⁺) was used for each organism and each set of tests. A sterility control and growth control were used for each organism to ensure there was no contamination and that growth occurred in the broth medium in the absence of GO-Ag⁺, respectively.

Table 5 presents results demonstrating the antimicrobial activity of the GO-Ag⁺ nanocomposite, reflected by the MICs for each of the AMR organisms tested, namely, Serratia marcescent (S. mar), Pseudomonas aeruginosa (P. aer 1), Pseudomonas aeruginosa (P. aer 2), Escherichia coli (ESBL), Escherichia coli (CRO), Klebsiella pneumonia (ESBL), Klebsiella pneumonia (CRO), Enterobacter aerogenes, Stenotrophomonas maltophilia, Methicillin Resistant Staphylococcus aureus (MRSA), CA-Methicillin Resistant Staphylococcus aureus (MRSA), Vancomycin Resistant Enterococcus faecium (VRE), Penicillin Resistant Streptococcus pneumoniae. The averaged MIC values (measured in μg/mL) of the GO-Ag⁺ nanocomposite against the exemplary organisms tested are presented. The MICs are based on complete inhibition in all 5 replicates where the “X” represents the lowest concentration of the GO-Ag⁺ nanocomposite at which no visible turbidity was observed.

The results of this evaluation demonstrate the efficacy of the GO-Ag⁺ nanocomposite against exemplary known AMR and MDR pathogens, including ESKAPE pathogens, which are associated with a number of difficult to treat clinical infections including those involving the respiratory tract, urinary tract, skin and soft tissues, and bacteremia at very low concentrations. The unexpectedly low MIC values ranged from 0.008 to 0.031 μg/mL (Table 5). Based on the previous evaluation of GO-Ag⁺ against fully susceptible organisms (i.e. E. coli, S. aureus, Streptococcus pneumoniae) (see Example 4), the MICs for the AMR strains were comparable or a single dilution higher.

TABLE 5
Broad- Spectrum Antimicrobial Activity (MIC) of GO-Ag⁺ Nanocomposite

| | Minimum Inhibitory Concentration (MIC, μg/mL) | | | | | | | | | Growth |
|---|---|---|---|---|---|---|---|---|---|---|
| Organism | 1 | 0.5 | 0.25 | 0.125 | 0.0625 | 0.031 | 0.0156 | 0.008 | 0.004 | Control |
| Gram Negatives | | | | | | | | | | |
| Serratia | | | | | | | X | | | Growth |
| marcesens | | | | | | | (0.0156) | | | |
| (S. mar) | | | | | | | | | | |
| Pseudomonas | | | | | | | X | | | Growth |
| aeruginosa | | | | | | | (0.0156) | | | |
| (P. aer 1) | | | | | | | | | | |
| Pseudomonas | | | | | | | X | | | |
| aeruginosa | | | | | | | (0.0156) | | | |
| (P. aer 2) | | | | | | | | | | |
| Escherichia | | | | | | X | | | | Growth |
| coli (ESBL) | | | | | | (0.031) | | | | |
| Escherichia | | | | | X | | | | | Growth |
| coli (CRO) | | | | | (0.0625) | | | | | |
| Klebsiella | | | | | | X | | | | Growth |
| pneumoniae | | | | | | (0.031) | | | | |
| (ESBL) | | | | | | | | | | |
| Klebsiella | | | | | X | | | | | Growth |
| pneumoniae | | | | | (0.0625) | | | | | |
| (CRO) | | | | | | | | | | |
| Enterobacter | | | X | | | | | | | Growth |
| aerogenes | | | (0.25) | | | | | | | |
| Stenotrophomonas | | | | | | X | | | | Growth |
| maltophilia | | | | | | (0.031) | | | | |
| Gram Positives | | | | | | | | | | |
| HA-Methicillin | | | | | X | | | | | Growth |
| Resistant | | | | | (0.0625) | | | | | |
| Staphylococcus | | | | | | | | | | |
| aureus (MRSA) | | | | | | | | | | |
| CA-Methicillin | | | | | | X | | | | Growth |
| Resistant | | | | | | (0.031) | | | | |
| Staphylococcus | | | | | | | | | | |
| aureus (MRSA) | | | | | | | | | | |
| Vancomycin | | | | | | X | | | | Growth |
| Resistant | | | | | | (0.031) | | | | |
| Enterococcus | | | | | | | | | | |
| faecium (VRE) | | | | | | | | | | |
| Penicillin | | | | | | | | X | | Growth |
| Resistant | | | | | | | | (0.008) | | |
| Streptococcus | | | | | | | | | | |
| pneumoniae | | | | | | | | | | |

### Example 6: Comparison of Antimicrobial Efficacy and Synergistic Effect

The antimicrobial efficacy of the GO-Ag⁺ nanocomposite, according to embodiments disclosed herein, was compared to the antimicrobial efficacy of previously described nanocomposites. As presented in Table 6, a comparison of the MICs of previously described nanocomposites, demonstrates an unexpected improved efficacy exhibited by the GO-Ag⁺ nanocomposite over previously described nanocomposites.

Table 6 presents a comparison of the antimicrobial activity of the GO-Ag⁺ nanocomposite for various pathogens compared to the antimicrobial efficacy of known metals and graphene composites as reported in: (a) Zhong, L. and Yun, K. “Graphene oxide-modified ZnO particles: synthesis, characterization antibacterial properties.” International Journal of Nanomedicine Spec. Iss. 10, 79-92; (b) Matar, Susan A. et al. “The antibacterial biofilm activity of metal-doped mullite ceramics against pathogenic bacteria.” African Journal of Microbiology Research 7 (23), June 2013, 2939-2947; (c) Salman, Halah Dawood, “Evaluation and Comparison the Antibacterial Activity of Silver Nano Particles (AgNPs) and Silver Nitrate (AgNO₃) on Some Pathogenic Bacteria.” Journal of Global Pharma Technology, December 2016; (d) Panacek et al. “Silver colloid nanoparticles: Synthesis, characterization and their antibacterial activity.” Journal of Physical Chemistry B 110 (33), 16248-16253; (e) Anni, Feng et al. “Facile Synthesis of Silver nanoparticles with High Antibacterial Activity.” Materials 11 (12), December 2018; (f) Ulkuseven, Bahri et al. “Synthesis, Characterization and antimicrobial Activity of d8-10 Metal Complexes of nine 2-substituted-1H-Benzimidazoles.” Metal-Based Drugs 6 (3), 1999; (g) Mazarin de Moraes, A. C. et al. “Graphene oxide-silver nanocomposite as a promising biocidal agent against methicillin-resistant Staphylococcus aureus.” Int J Nanomedicine 10:6847-6861, 2015.

Referring to the comparative data presented in Table 6, these data demonstrate that the GO-Ag⁺ nanocomposite, according to embodiments disclosed herein, significantly outperform graphene oxide (GO), graphene oxide-silver nanoparticle (AgNPGO), silver nitrate (AgNO₃), silver nanoparticles (AgNP), and zinc oxide-graphene oxide (ZnO.GO). As reflected by the MICs for representative pathogens, the MIC of the GO-Ag⁺ nanocomposite, according to embodiments disclosed herein, is at least 100× to 10,000× lower than the MIC of previously described nanocomposites.

Moreover, these data demonstrate the unexpected synergistic effect of graphene oxide with cationic silver (GO-Ag⁺ complex) when considering broad spectrum antimicrobial efficacy.

TABLE 6
Comparison of Antimicrobial Efficacy and Synergistic Effect

| | MIC (μg/mL) | | | | | |
|---|---|---|---|---|---|---|
| | | Silver | | | Zinc | Graphene |
| | | Nanoparticles- | | | Oxide- | Oxide- |
| | Graphene | Graphene | Silver | Silver | Graphene | Cationic |
| | Oxide | Oxide | Nitrate | Nanoparticles | Oxide | Silver |
| Organism | (GO) | (AgNPGO) | (AgNO₃) | (AgNP) | (ZnO•GO) | (GOAg⁺) |
| Streptococcus | | | 80 ^{c} | 50 ^{c} | | 0.0156 |
| pyogenes | | | | | | |
| Staphylococcus | >60 ^{g} | 256 ^{e} | 80 ^{c} | 60 ^{c} | | 0.031 |
| aureus | | | 50 ^{b} | 7 | | |
| | | 30 ^{g} | 9.8 ^{f} | 54 ^{d} | | |
| Moraxella | | | | | | 0.0156 |
| catarrhalis | | | | | | |
| Haemophilus | | | | | | 0.008 |
| influenzae | | | | | | |
| Escherichia | 12.5^{a} | 512 ^{e} | 130 ^{c} | 70 ^{c} | 6.25^{a} | 0.0625 |
| coli | | | | 3 | | |
| | >60 ^{g} | 15 ^{g} | 4.9 ^{f} | 27 ^{d} | | |
| Candida | | | 4.9 ^{f} | | | 0.0156 |
| albicans | | | | | | |
| Fusobacterium | | | | | | 0.0625 |
| nuclatum | | | | | | |
| Klebsiella | | | 140 ^{c} | 70 ^{c} | | |
| pneumoniae | | | 4.9 ^{f} | | | |
| Salmonella | 12.5^{a} | | 110 ^{c} | 70 ^{c} | 6.25^{a} | |
| typhi | | | | | | |
| Vibrio cholerae | | | 70 ^{c} | 50 ^{c} | | |
| Bacillus subtilis | 25^{a} | | | | 12.5^{a} | |
| Enterococcus | 50^{a} | 30 ^{g} | | | 25^{a} | |
| faecalis | >60 ^{g} | | | | | |
| Staphylococcus | >60 ^{g} | 15 ^{g} | | | | 0.031 |
| aureus (MRSA) | | | | | | |

^{a}Zhong, L. and Yun, K. “Graphene oxide-modified ZnO particles: synthesis, chracterization antibacterial properties.” International Journal of Nanomedicine Spec. Iss. 10, 79-92;
^{b} Matar, Susan A. et al. “The antibacterial biofilm activity of metal-doped mullite ceramics against pathogenic bacteria.” African Journal of Microbiology Research 7 (23), June 2013, 2939-2947;
^{c} Salman, Halah Dawood, “Evaluation and Comparison the Antibacterial Activity of Silver Nano Particles (AgNPs) and Silver Nitrate (AgNO₃) on Some Pathogenic Bacteria.” Journal of Global Pharma Technology, December 2016;
^{d} Panacek et al. “Silver colloid nanoparticles: Synthesis, characterization and their antibacterial activity.” Journal of Physical Chemistry B 110 (33), 16248-16253;
^{e} Anni, Feng et al. “Facile Synthesis of Silver nanoparticles with High Antibacterial Activity.” Materials 11 (12), December 2018;
^{f} Ulkuseven, Bahri et al. “Synthesis, Characterization and antimicrobial Activity of d8-10 Metal Complexes of soe 2-substituted-1H-Benzimidazoles.” Metal-Based Drugs 6 (3), 1999;
^{g} Mazarin de Moraes, A. C. et al. “Graphene oxide-silver nanocomposite as a promising biocidal agent against methicillin-resistant Staphylococcus aureus.” Int J Nanomedicine 10: 6847-6861, 2015.

### Example 7: Virucidal Activity for SARS-CoV-2

Virucidal activity of the GO-Ag⁺ nanocomposite was tested against SARS-CoV-2. Testing was also performed with GO-AgNP nanocomposite for comparison purposes.

### Preparation of GO-Ag⁺ Nanocomposite

GO-Ag⁺ nanocomposite was formulated as a suspension. A mixture of ethanol and deionized water (DI) was used. In 100 mL of suspension, 60 to 70 mL of ethanol and 30 to 40 mL of DI water were used as the diluent. The GO-Ag⁺ nanocomposite was suspended in the diluent at a concentration of 0.1 to 5 g/L making a dispersion. The resulting formulation comprising GO-Ag⁺ nanocomposite was applied to surfaces and filter media by either dip or spray coating and then air-dried or thermally-dried to fix the GO-Ag⁺ nanocomposite onto the media.

The materials were prepared 5 weeks prior to virucidal testing. The materials were autoclaved to sterilize before testing at 121° C. for 30 min prior to the test analyses. After sterilization, inside a biological safety cabinet (BSC), the material was cut into ˜0.5×0.5 cm squares and placed into sterile 1.5 ml tubes.

### SARS-CoV-2 Preparation and Testing

The SARS-COV-2 virus stock at a titer of 10^{5.8} infectious units (IU)/ml was diluted to 102.9 (IU)/ml. A volume of 500 ml of the diluted viral stock was added to a 1.5 ml tube containing the square of the coated material. The tube containing the virus and coated material was placed on a tube rotator for 2 hrs at 22° C. Untreated material (0-001-011) exposed to viral supernatant was used as a control. Collected supernatants were diluted 100-fold to dilute any chemicals/materials that may have been released from the coated material during incubation. The collected supernatants (diluted 100-fold) were further serially diluted from 1:100 to 1:100,000 and then added to 20,000 Vero E6 cells in 96 well flat-bottom plates.

The 1:100 dilution of the virus stock infecting 20,000 cells represents a multiplicity of infection (MOI) of 0.02. Infection of the Vero E6 cells was monitored by viral cytotoxicity. Cell toxicity of the supernatants-derived material in the absence of virus (diluted 1:100) was measured visually.

### Results

As shown in Table 7, viral titers were reduced by 2 Logs corresponding to 99% reduction in infectious virus after 2 hours of exposure to the GO-Ag⁺ nanocomposite coated material. Exposure to the untreated control material resulted in no reduction of viral titers. All experiments were performed in triplicate. Variance in the results was less than 5% and reported as 99% reduction in infectious virus (or 99% in viricidal activity).

TABLE 7
Virudical Activity of GO-Ag⁺ (Cation) Nanocomposite
on SARS-CoV-2 Infected VERO E6 Cells

| | | Reduction Factor | |
|---|---|---|---|
| Sample N-95 | Viral | (Log10) v. Control | % Viral |
| Material | Titer | (Post 2 HRs Exposure) | Reduction |
| GO-Ag+ Sample 1 | 10^{2.8} | 2 | 99% |
| GO-Ag+ Sample 2 | 10^{2.8} | 2 | 99% |
| Untreated Control | 10^{3.8} | 0 | 0% |

*Diluted control virus after dilution resulted in a TCID50/ml of 10^{3.8}

### Preparation of GO-AgNP Nanocomposite

To synthesize Ag-doped GO nanosheets, 0.1 g dried pristine GO powder was dispersed in 30 mL DI water in a 100 mL Erlenmeyer flask using a bath sonicator for 30 min. The pH of the GO suspension was adjusted at 10 using NaOH solution (0.1 M). Then, 2 mL of the AgNO₃ solution (0.25 M) was added to the previous suspension under stirring (400 rpm). Next, 20 mL DI water was added to the previous suspension to reduce the viscosity of the solution. The mixture was stirred for 20 h at 60° C.

### Method for SARS-COV-2 Replication Inhibition Tests

The virucidal activity of GO-AgNP nanocomposite was tested at the ImPaKt Facility at Western University. The GO-AgNP nanocomposite was in the form of a thick viscosity paste which was applied to the bottom surface of a 12-well dish using a flat edge weight spoon.

SARS-CoV-2 Wuhan strain viruses were serially diluted 4 times to produce infectious units of approximately 200,000 infectious units (IU), 20,000 IU, 2,000 IU, and 200 IU per 20 μL. 200 μL of the SARS-COV-2 dilutions were overlaid onto the GO-AgNP treated and untreated surfaces. After 1 hour and 12 hours incubation of virus with the treated surfaces, 20 μL of supernatant in each well was added to wells of a new 96 well plates containing approximately 20,000 Vero cells in DMEM media. The final multiplicity of infection was 2.0, 0.2, 0.02, and 0.002 infectious units per cell for each viral dilution. Viral cytopathic effects (vCPE) on cells were observed within a day and vCPE was measured at day 3.

Results from SARS-CoV-2 Replication Inhibition Tests

The results of the replication/inhibition tests are presented in Table 8.

At 2,000 IU, the GO-AgNP nanocomposite had approximately 20-30% protective effect on the SARS-COV-2 infection of VERO E6 cells. At both 200 IU and 20 IU, approximately 10-20% protective effect was observed on the SARS-COV-2 infection of VERO E6 cells. At 20,000 IU of virus, no protective effect was observed. At 12 hours, clear viral activity was significantly decreased in both treated and untreated conditions. No difference was observed between treated and untreated surfaces.

TABLE 8
Viral Cytopathic Effect (vCPE) of GO-AgNP nanocomposite
on SARS-CoV-2 Infected VERO E6 Cells
P2 - 1 hr treatment

| | | | | |
|---|---|---|---|---|
| 20,000 | IU | +++++ | +++++ | +++++ |
| 2,000 | IU | +++ | +++ | +++ |
| 200 | IU | +++ | +++ | +++ |
| 20 | IU | ++ | ++ | ++ |

| | | | |
|---|---|---|---|
| 0 | N.I. | N.I. | N.I. |

1 hr - no treatment

| | | | | |
|---|---|---|---|---|
| 20,000 | IU | +++++ | +++++ | +++++ |
| 2,000 | IU | +++++ | +++++ | +++++ |
| 200 | IU | ++++ | ++++ | ++++ |
| 20 | IU | ++++ | ++++ | ++++ |

| | | | |
|---|---|---|---|
| 0 | N.I. | N.I. | N.I. |

Key:
+++++ 80-100% Infected cells
++++ 60-80% Infected cells
+++ 40-60% Infected cells
++ 20-40% Infected cells
+ 1-10% Infected cells
N.I. 0% Infected cells

### Example 8: Tolerated Dose of Graphene-Silver Cation Nanocomposite—Range Finding Study

Acute toxicity studies were conducted to determine the short-term adverse effects of the GO-Ag⁺ nanocomposite. An initial single dose acute toxicity study was conducted to provide information on the potential for acute toxicity in humans, estimate safe acute doses for humans, identify the potential target organs of toxicity, and estimate the appropriate dosage for multiple-dose toxicity studies.

3 naïve male and 3 naïve female Sprague-Dawley rats were randomly assigned to the study. The animals received dose formulation containing the GO-Ag⁺ nanocomposite suspended in high viscosity 1% methyl cellulose into the oral cavity on the back of the tongue close to the rat's throat. The control item (vehicle) used in this study was 1% methyl cellulose.

The maximum tolerated dose (MTD) of the GO-Ag⁺ nanocomposite following the single oral cavity dose was determined starting with 1000 mg/kg as outlined in Table 9. The GO-Ag⁺ nanocomposite was administered to groups of 3 males and 3 female rats following an up-and-down procedure as described in Table 9.

TABLE 9
Study Outline

| | | | | Observation |
|---|---|---|---|---|
| | | | | Period for |
| Dose | Dose Level | Dose Volume | Number of | Surviving |
| Sequence | (mg/kg) | (mL/kg) | Animals | Animals |
| 1^{st} | 1000 | 3.33 | 3M/3F | 7 days |
| 2^{nd} | 2000 | 5.714 | 3M/3F | 7 days |

M = Male;
F = Female

As the first group of animals survived and did not show toxic effect, the second group of animals received a higher dose. Based on the reaction of the previous group, the following group was dosed at 24 to 72-hour intervals.

Mortality checks and clinical observations were conducted twice daily. Body weights were measured prior to dosing on Day 1 and prior to necropsy on Day 8. Food consumption was measured weekly. Full gross pathology was performed on all surviving animals. The necropsy consists of an external examination including reference to all clinically recorded lesions, as well as a detailed internal examination.

### Results and Conclusions

All animals at both dose levels survived, appeared normal, and gained weight when the GO-Ag⁺ nanocomposite was administered at the dose level of 1000 mg/kg and 2000 mg/kg and throughout the 7-day observation period. There were no abnormal findings noted upon gross necropsy.

### Example 9: Toxicokinetics (TK) of Graphene-Silver Cation Nanocomposite—7-Day Repeated Dose Study

A 7-day repeated dose toxicity study of the GO-Ag⁺ nanocomposite was conducted to evaluate toxicity following 7 consecutive days of dosing when administered in the oral cavity of mice. TK studies were carried out to determine any toxic effects from exposure (AUC and Cmax) to the GO-Ag⁺ nanocomposite.

The GO-Ag⁺ nanocomposite and vehicle control were administered daily for 7 consecutive days via oral cavity administration as outlined in Table 10. The GO-Ag⁺ nanocomposite formulations were prepared by suspension in high viscosity 1% methyl cellulose. The control item (vehicle) used in this study was 1% methyl cellulose. GO-Ag⁺ nanocomposite dosing formulations and vehicle control were administered by placing the dose volume on the back of the tongue using a syringe with a blunt tip gavage needle. The dose was delivered slowly drop-wise to the oral cavity and throat area.

TABLE 10
7-Day Repeated Dose and TK Study

| | Dose | Number of Animals | | |
|---|---|---|---|---|
| Group | (mg/kg/day) | Main Study | TK Study | Comments |
| 1. Control | 0 | 4M/4F | — | Daily oral cavity |
| 2. Low Dose | 50 | 4M/4F | 3M/3F | Dosing and |
| 3. Mid Dose | 250 | 4M/4F | 3M/3F | Observations |
| 4. High Dose | 1000 | 4M/4F | 3M/3F | for 7 days |

Mortality checks and clinical observations were conducted twice daily. Body weights were recorded prior to dosing on Day 1 and Day 7 and on Day 8 prior to necropsy. Food was measured weekly. Clinical pathology evaluations (hematology and clinical chemistry) were performed prior to necropsy. Blood samples were collected from 6 animals per treatment group per time point for toxicokinetic analysis on Days 1 and 7 at 1, 2, 4, and 8 hours post-dose. Full gross pathology was performed on all Main Study animals. Organ weights were recorded for all Main Study animals. Histopathology was performed on a comprehensive range of organs from all Main Study animals.

### Results and Conclusions

### Clinical Signs

There were no abnormal clinical observations noted during the 7-day repeated dose study at 50, 250 and 1000 mg/kg/day with the exception of dark feces noted in 1000 mg/kg/day group during the last four days of treatment.

### Body Weights and Food Consumption

There were no significant differences observed in body weight gains or food consumption between the groups. However, the low dose and high dose males were observed to consume less food than the control group over the study period and, as presented in Table 11, there was an insignificantly lower weight gain in the low, mid and high dose males compared to the control group.

Table 11 presents a summary of body weights and food consumption observed in a 7-day repeated dose of GO-Ag⁺ nanocomposite at a Low Dose of 50 mg/kg/day (2-M, 2-F), Mid Dose of 250 mg/kg/day (3-M, 3-F), and High Dose 1000 mg/kg/day (4-M, 4-F) in four male and four female rats against a 1% methyl cellulose as Control (1-M, 1-F).

TABLE 11
Summary of Body Weights and Food Consumption

| | | | Mean Body | |
|---|---|---|---|---|
| | | | Weight | Mean Total |
| | | | Change ± | Food |
| | Mean Body Weight ± S.D. (g) | | S.D. (g) | Consumption ± |
| Group | Day 1 | Day 7 | (Day 1-7) | S.D. (g) |
| 1-M | 231.7 ± 10.7 | 275.8 ± 12.0 | +44.2 ± 3.1 | 147.4 ± 5.3 |
| 2-M | 234.2 ± 11.7 | 266.8 ± 12.6 | +32.6 ± 9.1 | 131.0 ± 7.0* |
| 3-M | 232.2 ± 10.8 | 260.2 ± 14.7 | +28.0 ± 9.1 | 134.8 ± 4.3 |
| 4-M | 228.9 ± 13.1 | 258.2 ± 23.1 | +29.3 ± 15.5 | 129.5 ± 13.4* |
| 1-F | 191.2 ± 9.7 | 212.6 ± 13.4 | +21.4 ± 5.4 | 110.9 ± 8.5 |
| 2-F | 193.8 ± 11.1 | 215.1 ± 16.9 | +21.2 ± 11.8 | 113.6 ± 14.0 |
| 3-F | 195.2 ± 7.2 | 210.8 ± 13.9 | +15.6 ± 8.2 | 116.0 ± 11.0 |
| 4-F | 191.0 ± 14.7 | 211.3 ± 19.3 | +20.3 ± 6.6 | 107.6 ± 15.3 |

n = 4
*statistically significant from control (p < 0.05)/

### Clinical Pathology

Clinical pathology investigations (hematology and clinical chemistry) were performed on Day 8. There were no findings in blood clinical pathology that could be attributed to the treatment with GO-Ag⁺ nanocomposite.

### Toxicokinetics (TK)

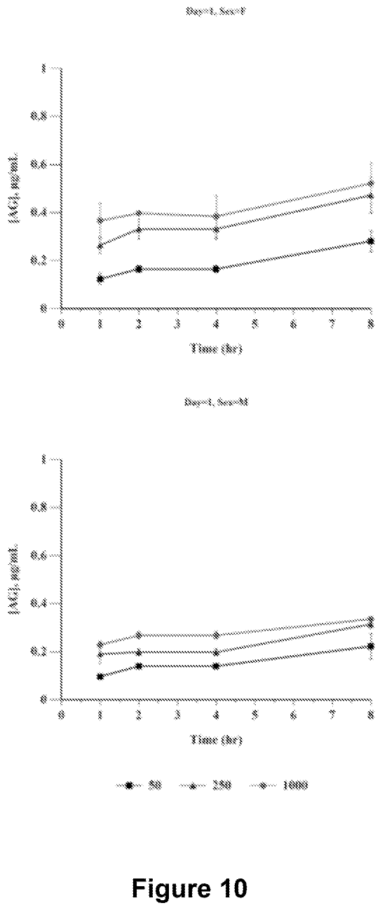

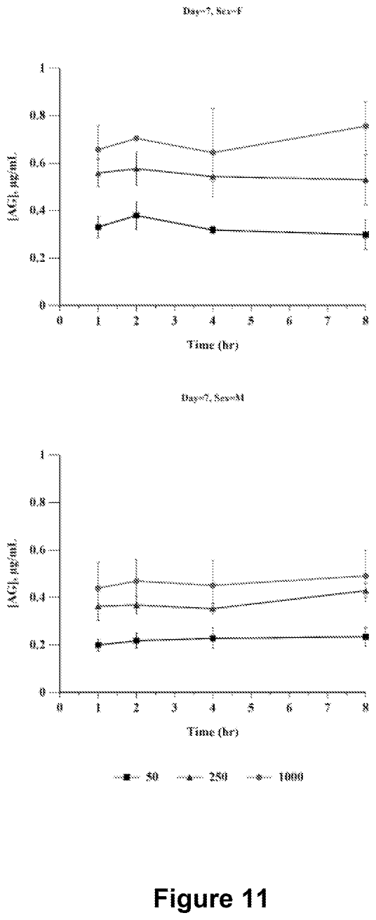

Maximum plasma concentrations of Ag on Day 1 ranged from 0.22 to 0.34 μg/mL in males and 0.26 to 0.50 μg/mL in females (FIG. 10) and there were no differences between male and female rats on Days 1 and 7 (FIG. 11). There was a slight increase in Cₘₐₓ in both male and female rats on day 7 compared to Day 1 at mid and high dose levels. Time to maximum plasma concentrations as measured by Tₘₐₓ was similar for all dose levels in both male and female rats on Day 1 (8 hours) after single dose administration. Average Tₘₐₓ after seven repeated doses was shorter in both male and female rats with 6.1 and 3.3 hours respectively, on Day 7. Table 12 shows the pharmacokinetic parameters of Ag in male and female rats following oral dosing with GO-Ag⁺ nanocomposite.

The plasma exposure to Ag as measured by AUC₀₋ₗₐₛₜ ranged from 1.22 and 2.22 hr*μg/mL in males and 1.80 and 3.70 hr*μg/mL in females. Both male and female rats exhibited slightly higher AUC₀₋ₜₗₐₛₜ on day 7 compared to Day 1 only at mid and high dose levels, although not a substantially higher accumulation (Table 12).

TABLE 12
Pharmacokinetic Parameters for Ag in Male
and Female Rats Following Oral Dosing

| | | | |
|---|---|---|---|
| | Cₘₐₓ | Tₘₐₓ | AUC_{0-Tlast} |
| Dose of Ag | μg/mL | hr | hr*μg/mL |

Males Day 1

| | | | | |
|---|---|---|---|---|
| 50 | mg/kg | 0.22 | 8 | 1.22 |
| 250 | mg/kg | 0.31 | 8 | 1.80 |
| 1000 | mg/kg | 0.34 | 8 | 2.22 |

Males Day 7

| | | | | |
|---|---|---|---|---|
| 50 | mg/kg | 0.26 | 4.7 | 1.8 |
| 250 | mg/kg | 0.43 | 8.0 | 3.0 |
| 1000 | mg/kg | 0.50 | 5.7 | 3.7 |

Females Day 1

| | | | | |
|---|---|---|---|---|
| 50 | mg/kg | 0.28 | 8 | 1.49 |
| 250 | mg/kg | 0.47 | 8 | 2.83 |
| 1000 | mg/kg | 0.59 | 8 | 3.46 |

Females Day 7

| | | | | |
|---|---|---|---|---|
| 50 | mg/kg | 0.38 | 2 | 2.60 |
| 250 | mg/kg | 0.60 | 6 | 4.36 |
| 1000 | mg/kg | 0.70 | 2 | 5.14 |

Table 13 summarizes the dose proportionality of Ag mean Cₘₐₓ and AUC values from oral dosing with GO-Ag+ nanocomposite. As shown in Table 13, the ratio of Cₘₐₓ and AUC₀₋ₜₗₐₛₜ between mid to low dose and high to low doses was surprisingly low at about <2 in comparison to the dose ratios of 5 and 20 fold.

TABLE 13
Dose Proportionality of Ag mean Cₘₐₓ and AUC Values from Oral Dosing

| | Dose | Dose | Cₘₐₓ | Cₘₐₓ | AUC [symbol] | AUC [symbol] |
|---|---|---|---|---|---|---|
| | Ratio | Ratio | Ratio | Ratio | Ratio Mid | Ratio High |
| | Mid to | High to | Mid to | High to | to Low | to Low |
| Study Days/Sex | Low Dose | Low Dose | Low Dose | Low Dose | Dose | Dose |
| Day 1/Males | 5 | 20 | 1.41 | 1.51 | 1.48 | 1.82 |
| Day 7/Males | | | 1.67 | 1.94 | 1.69 | 2.08 |
| Day 1/Females | | | 1.68 | 2.09 | 1.90 | 2.32 |
| Day 7/Females | | | 1.58 | 1.86 | 1.68 | 1.98 |

[symbol]

indicates data missing or illegible when filed

Table 14 shows the comparison of Ag Cₘₐₓ and AUCₜₗₐₛₜ between Day 1 and Day 7 and male and females following oral dosing with GO-Ag⁺ nanocomposite. Comparison of Day 1 and Day 7 Cₘₐₓ and AUC₀₋ₜₗₐₛₜ values similarly did not present dramatic increases as what would be expected at the doses tested (Table 14).

TABLE 14
Comparison of Ag Cₘₐₓ and AUCₜₗₐₛₜ between Day 1 and
Day 7 and Male and Female Following Oral Dosing

| | | | Female | | | Male | |
|---|---|---|---|---|---|---|---|
| Dose | Study | Rat | Cₘₐₓ | AUC_{0-Tlast} | Rat | Cₘₐₓ | AUC_{0-Tlast} |
| of Ag | Days | ID | μg/mL | hr* μg/mL | ID | μg/mL | hr* μg/mL |
| 50 | Day 1 | 34 | 0.32 | 1.58 | 9 | 0.17 | 1.07 |
| mg/kg | | 35 | 0.29 | 1.41 | 10 | 0.23 | 1.27 |
| | | 36 | 0.23 | 1.48 | 11 | 0.27 | 1.32 |
| | Day 7 | 34 | 0.44 | 2.88 | 9 | 0.22 | 1.66 |
| | | 35 | 0.34 | 2.27 | 10 | 0.28 | 1.99 |
| | | 36 | 0.36 | 2.65 | 11 | 0.28 | 1.68 |
| 250 | Day 1 | 41 | 0.47 | 2.66 | 16 | 0.31 | 1.66 |
| mg/kg | | 42 | 0.40 | 2.89 | 17 | 0.30 | 1.83 |
| | | 43 | 0.55 | 2.95 | 18 | 0.34 | 1.92 |
| | Day 7 | 41 | 0.63 | 4.61 | 16 | 0.43 | 3.08 |
| | | 42 | 0.65 | 4.25 | 17 | 0.40 | 2.73 |
| | | 43 | 0.55 | 4.22 | 18 | 0.46 | 3.21 |
| 1000 | Day 1 | 48 | 0.59 | 3.46 | 23 | 0.33 | 2.22 |
| mg/kg | | | | | 24 | 0.33 | 2.12 |
| | | | | | 25 | 0.35 | 2.32 |
| | Day 7 | 48 | 0.70 | 5.14 | 23 | 0.42 | 3.16 |
| | | | | | 24 | 0.46 | 3.28 |
| | | | | | 25 | 0.61 | 4.63 |

### Organ Weights and Pathology

There were no significant or clinically relevant alterations in absolute organ weights, organ/body weight or organ/brain weight ratios. There were no abnormal findings from histopathology attributed to treatment with GO-Ag⁺ nanocomposite.

### CONCLUSIONS

In conclusion, analysis of all generated data indicated that the GO-Ag⁺ nanocomposite was well tolerated following a 7-day repeated oral dose administration at the dose levels of 50, 250, and 1000 mg/kg.

### Example 10: In Vivo Studies—Topical Antimicrobial Efficacy

The in vivo efficacy of the GO-Ag⁺ nanocomposite was tested on a small scale in human subjects to observe the broad-spectrum antimicrobial effect against different conditions.

### Preparation of GO-Ag⁺ Nanocomposite Formulation

The GO-Ag⁺ nanocomposite was formulated as a 1 g/L GO-Ag⁺ nanocomposite water suspension, i.e., a 0.1% GO-Ag⁺ nanocomposite suspension in water, as well as a 0.4% GO-Ag⁺ nanocomposite-PEG cream formulation comprising 80% (PEG 400) and 20% (PEG 3350) by volume.

### Topical Application

The formulation was topically applied to the area identified for treatment by either spraying the water suspension directly on the affected area or applying the PEG cream formulation to the affected area using an applicator or finger. For both formulations (water suspension and PEG cream), the GO-Ag⁺ nanocomposite formulation was rubbed into the skin until no longer visible.

### Indications Tested

Indications were chosen to observe the broad-spectrum efficacy demonstrated in the in vitro studies. In particular, indications included those caused by fungal or bacterial infections including acne, sebhorreic dermatitis and toe nail fungus. The observations of the treatment are presented in Table 15.

TABLE 15
Topical Treatment with GO-Ag⁺ Nanocomposite Formulation - Observations

| | Subject | | | | | Adverse |
|---|---|---|---|---|---|---|
| ID | Demographic | Indication | Formulation | Treatment | Result | Reaction |
| 1 | 26-year | moderate | Water | two | substantial | none |
| | old male | seborrheic | suspension | applications | clearing of | |
| | | dermatitis | | sprayed on | the erythematous | |
| | | | | affected | scaling plaques | |
| | | | | area | within 48 hours | |
| 2 | 31-year | acneiform | Water | sprayed on | postular | none |
| | old female | folliculitis | suspension | affected | eruption | |
| | | in the neck | | area, twice | was 75% | |
| | | and “facemask | | daily | improved | |
| | | area” | | | | |
| 3 | 54-year | acne | Water | sprayed on | reduction of | none |
| | old female | | suspension | affected | acne notable | |
| | | | | area, once | increase in | |
| | | | | daily | breakouts | |
| | | | | | when treatment | |
| | | | | | was stopped | |
| 4 | 22-year | comedonal | Water | sprayed on | no effect | none |
| | old female | acne | suspension | affected | | |
| | | | | area, twice | | |
| | | | | daily for | | |
| | | | | 10 days | | |

### Results

### Acne

Treatment of some forms of acne was shown to be effective. In particular, two subjects (ID 2 and 3) reported up to a 75% improvement in eruptions. On the other hand one subject (ID 4) with comedonal acne did not experience any benefit from treatment. An explanation may be that comedonal acne is caused by increased sebum production and blockage of the sebaceous duct or hair follicle which would not be responsive to the antimicrobial effect of the GO-Ag⁺ nanocomposite formulation. In contrast, the acne in the other two subjects, i.e., acneiform folliculitis, is caused by bacterial infection (e.g., Staphylococcus aureus, Pseudomonas aeruginosa, Malassezia, resistant gram-negative folliculitis) and showed improvement with treatment with the GO-Ag⁺ nanocomposite formulation.

### Seborrheic Dermatitis

Seborrheic dermatitis may be caused by a number of agents including a Malassezie yeast. A 26-year old male subject (ID 1) presented with a moderate case of seborrheic dermatitis which is a red inflammation of the skin. The condition was substantially cleared within 48 hours after treatment indicating antimicrobial efficacy of the GO-Ag⁺ nanocomposite formulation to improve seborrheic dermatitis.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1-61: (canceled)

62. A method of treating a microbial infection in a subject comprising administering to the subject a therapeutically effective amount of an antimicrobial nanocomposite comprising graphene oxide (GO) and silver cations (Ag⁺) bound to the GO as Ag(1)-complexes.

63. The method according to claim 62, wherein the silver cations (Ag⁺) bound to the GO are bound by complex bonds.

64. The method according to claim 63, wherein the complex bond is a chelated bond or a coordinate covalent bond.

65. The method according to claim 62, wherein the nanocomposite comprises between 3-80% w/w, 10-20% w/w, or 4-8% w/w of silver cations bound to the GO.

66. The method according to claim 62, further comprising silver nanoparticles covalently bound to the GO of the nanocomposite.

67. The method according to claim 66, wherein the ratio of silver cation to silver nanoparticle ranges from a ratio of 10:1 to 15:1.

68. The method according to claim 66, wherein the silver bound to the GO comprises about 90-99% silver cations (Ag⁺) in an Ag(1)-complex form and about 1-10% silver nanoparticles in a clustered Ag(0)-nanoparticle form.

69. The method according to claim 62, further comprising copper cations (Cu²⁺), or zinc cations (Zn²⁺).

70. The method according to claim 62, wherein the nanocomposite has a particle size ranging from 2 to 10 μm.

71. The method according to claim 62, wherein the microbial infection is selected from the group consisting of Pseudomonas aeruginosa, Escherichia coli, Klebsiella pneumoniae, marcescens, Serratia Acinetobacter baumanii, Stenotrophomonas maltophilia, Streptococcus pneumonia, Staphylococcus aureus, Candida Auris, influenza virus, Extended Spectrum Beta-lactamase (ESBL) Escherichia coli, ESBL Klebsiella pneumoniae, Carbapenem Resistant Organisms (CRO) Enterobacter spp., Penicillin-resistant Streptococcus pneumonia, CA-MRSA, HA-MRSA, and Acinetobacter baumanii complex.

72. The method according to claim 62, wherein the microbial infection is a bacterial infection, a viral infection, or a fungal infection.

73. The method according to claim 72, wherein the viral infection is an influenza virus, flavivirus, parainfluenza virus, respiratory syncytial virus, coronavirus, adenovirus, rhinovirus infection, or SARS-COV-2 virus.

74. The method according to claim 72, wherein the bacterial infection is a Corynebacterium diphtheria, Neisseria gonorrhoeae, Mycoplasma pneumonia, Mycoplasma hominis, Haemophilus influenzae, Streptococcus pnemoniae, Mycoplasma pnemoniae, Streptococcus pnemoniae, Staphylococcus aureus, Streptococcus pyogenes, Klebsiella pneumonia, Escherichia coli, Pseudomonas aeruginosa, Legionella spp, Mycobacterium tuberculosis, Coxiella burnetii, or Chlamydophila pneumoniae infection.

75. The method according to claim 72, wherein the fungal infection is a Candida albicans, Aspergillus spp, Histoplasma capsulatum, Blastomyces dermitidis, Paracoccidioides brasifiensis, or Coccidioides immis infection.

76. The method according to claim 62, wherein the microbial infection is a respiratory tract infection.

77. The method according to claim 76, wherein the respiratory tract infection is rhinitis, sinusitis, pharyngitis, epiglottitis, laryngitis, bronchitis, bronchiolitis, or pneumonia.

78. The method according to claim 76, wherein the nanocomposite is administered into the airway, bronchus or lungs via an intranasal or an inhalation route.

79. The method according to claim 62, wherein the microbial infection is a multidrug resistant infection.

80. The method according to claim 79, wherein the multidrug resistant infection is a Pseudomonas aeruginosa, Escherichia coli, Klebsiella pneumoniae, Serratia marcescens, Acinetobacter baumanii, Stenotrophomonas maltophilia, Streptococcus pneumonia, Staphylococcus aureus, Extended Spectrum Beta-lactamase (ESBL) Escherichia coli, ESBL Klebsiella pneumoniae, Carbapenem Resistant Organisms (CRO) Enterobacter spp., Penicillin-resistant Streptococcus pneumonia, CA-MRSA, HA-MRSA, Acinetobacter baumanii complex, an ESKAPE pathogen selected from the group consisting of Enterococcus faecium, Staphylococcus aureus, Klebsiella pneumoniae, Acinetobacter baumannii, Pseudomonas aeruginosa, and Enterobacter spp, or Candida auris.

81. The method according to claim 62, wherein the nanocomposite is formulated as a powder, a liquid, or an ointment.
